(19) United States
(12) Patent Application Publication
(10) Pub. No.: US 2024/0139162 A1
(43) Pub. Date: May 2, 2024

(54) Benzophenanthridine Alkaloids and Their Methods of Use

(71) Applicant: Chengdu Anticancer Bioscience, LTD., Chengdu (CN)

(72) Inventors: Dun Yang, Chengdu (CN); Jing Zhang, Chengdu (CN); Shenqiu Zhang, Chengdu (CN); Jinhua Li, Chengdu (CN); Qiong Shi, Chengdu (CN)

(21) Appl. No.: 17/769,415

(22) PCT Filed: Oct. 16, 2020
(86) PCT No.: PCT/CN2020/121431

§ 371 Date: Apr. 15, 2022

(30) Foreign Application Priority Data
Oct. 17, 2019 (WO) PCT/CN2019/111565

(51) Int. Cl. A61K 31/4355

(52) U.S. Cl. CPC A61K 31/4355

(57) ABSTRACT

Described herein are compounds that can arrest mitotic cells and their use in the treatment of disorders such as cancers.

### FIELD OF INVENTION

The present invention relates in part to benzophenanthridine alkaloid compounds, compositions comprising benzophenanthridine alkaloid compounds, and methods for using such benzophenanthridine alkaloid compounds to treat cancer.

### BACKGROUND OF THE INVENTION

Cancer is the second leading cause of death globally and was responsible for an estimated 9.6 million deaths in 2018. According to the American Cancer Society, at least one third of these individuals are not expected to survive the disease, highlighting the need for new and innovative treatments. Systemic chemotherapy remains the standard of care for cancer treatment, and agents that disrupt mitotic spindle assembly—so called “anti-mitotics”—are commonly used to treat a wide variety of cancers. Traditional anti-mitotic agents include the microtubule toxins such as taxol, other taxanes, and the vinca alkaloids, all of which have proven successful in the clinic. However, patient response remains highly unpredictable and drug resistance is common. Additionally, off-target toxicity is a problem with these broad-acting agents.

Safer and more efficacious compounds with anti-mitotic activity are needed for cancer treatment.

### SUMMARY OF THE INVENTION

Provided herein is a method of treating cancer in a subject in need thereof, the method comprising: administering to the subject in need thereof a therapeutically effective amount of a compound of Formula I

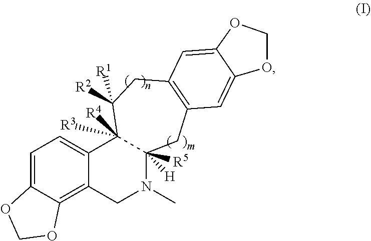

wherein: - - - denotes no bond or a single bond; R¹ is H; R² is selected from —OH and —OC(O)CH₃; or R¹ and R² are joined together to form, together with the carbon to which they are attached, an oxo; R³ is selected from H and CH₃; R⁴ is H or absent; R⁵ is H or absent; n is selected from 0 and 1; m is selected from 0 and 1; and provided that: when - - - denotes no bond, both R⁴ and R⁵ are H; when - - - denotes a single bond, both R and R⁵ are absent; when n is 0, R¹ and R² are joined together to form an oxo, R³ is H, both R⁴ and R⁵ are H, - - - is no bond, and m is 1.

Also provided herein is a method of disrupting mitotic spindle assembly in a cell in a subject, the method comprising: administering to the subject in need thereof a therapeutically effective amount of a compound of Formula I

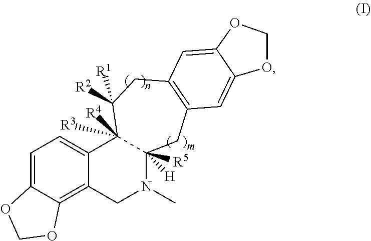

wherein: - - - denotes no bond or a single bond; R¹ is H; R² is selected from —OH and —OC(O)CH₃; or R¹ and R² are joined together to form, together with the carbon to which they are attached, an oxo; R³ is selected from H and CH₃; R⁴ is H or absent; R⁵ is H or absent; n is selected from 0 and 1; m is selected from 0 and 1; and provided that: when - - - denotes no bond, both R⁴ and R⁵ are H; when - - - denotes a single bond, both R⁴ and R⁵ are absent; when n is 0, R¹ and R² are joined together to form an oxo, R³ is H, both R⁴ and R⁵ are H, - - - is no bond, and m is 1.

Also provided herein is a method of blocking completion of cytokinesis in a cell in a subject, the method comprising: administering to the subject in need thereof a therapeutically effective amount of a compound of Formula I

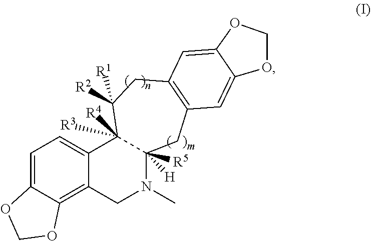

wherein: - - - denotes no bond or a single bond; R¹ is H; R² is selected from —OH and —OC(O)CH₃; or R¹ and R² are joined together to form, together with the carbon to which they are attached, an oxo; R³ is selected from H and CH₃; R⁴ is H or absent; R⁵ is H or absent; n is selected from 0 and 1; m is selected from 0 and 1; and provided that: when - - - denotes no bond, both R⁴ and R⁵ are H; when - - - denotes a single bond, both R⁴ and R⁵ are absent; when n is 0, R¹ and R² are joined together to form an oxo, R³ is H, both R⁴ and R⁵ are H, - - - is no bond, and m is 1. In some aspects, completion but not initiation of cytokinesis is blocked.

Also provided herein is a method of inducing tumor cell apoptosis in a subject, the method comprising: administering to the subject in need thereof a therapeutically effective amount of a compound of Formula I

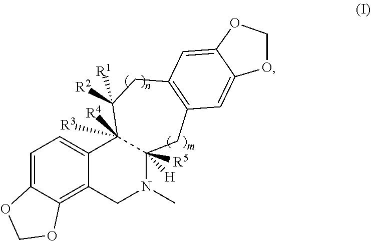

wherein: - - - denotes no bond or a single bond; R¹ is H; R² is selected from —OH and —OC(O)CH₃; or R¹ and R² are joined together to form, together with the carbon to which they are attached, an oxo; R³ is selected from H and CH₃; R⁴ is H or absent; R⁵ is H or absent; n is selected from 0 and 1; m is selected from 0 and 1; and provided that: when - - - denotes no bond, both R⁴ and R⁵ are H; when - - - denotes a single bond, both R⁴ and R⁵ are absent; when n is 0, R¹ and R² are joined together to form an oxo, R³ is H, both R⁴ and R⁵ are H, - - - is no bond, and m is 1.

In an embodiment, provided herein is a method of arresting cell mitosis in a cell in a subject, the method comprising: administering to the subject in need thereof a therapeutically effective amount of a compound of Formula I

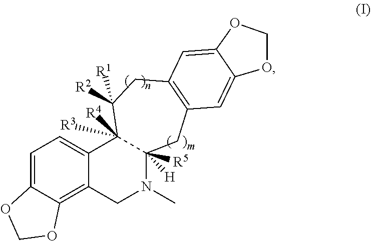

wherein: - - - denotes no bond or a single bond; R¹ is H; R² is selected from —OH and —OC(O)CH₃; or R¹ and R² are joined together to form, together with the carbon to which they are attached, an oxo; R³ is selected from H and CH₃; R⁴ is H or absent; R⁵ is H or absent; n is selected from 0 and 1; m is selected from 0 and 1; and provided that: when - - - denotes no bond, both R⁴ and R⁵ are H; when - - - denotes a single bond, both R⁴ and R⁵ are absent; when n is 0, R¹ and R² are joined together to form an oxo, R³ is H, both R⁴ and R⁵ are H, - - - is no bond, and m is 1.

In another embodiment, provided herein is a method of modulating mitotic index of a cell in a subject, the method comprising: administering to the subject in need thereof a therapeutically effective amount of a compound of Formula I

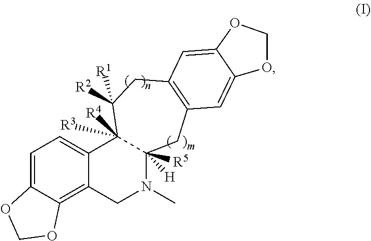

wherein: - - - denotes no bond or a single bond; R¹ is H; R² is selected from —OH and —OC(O)CH₃; or R¹ and R² are joined together to form, together with the carbon to which they are attached, an oxo; R³ is selected from H and CH₃; R⁴ is H or absent; R⁵ is H or absent; n is selected from 0 and 1; m is selected from 0 and 1; and provided that: when - - - denotes no bond, both R⁴ and R⁵ are H; when - - - denotes a single bond, both R⁴ and R⁵ are absent; when n is 0, R¹ and R² are joined together to form an oxo, R³ is H, both R⁴ and R⁵ are H, - - - is no bond, and m is 1.

In some embodiments, provided herein is a method of modulating a mitotic regulator, comprising administering an effective amount of a compound of Formula I

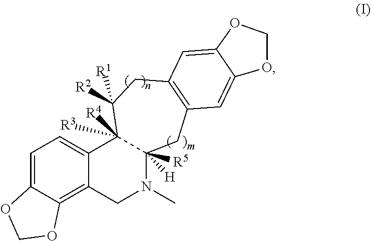

wherein: - - - denotes no bond or a single bond; R¹ is H; R² is selected from —OH and —OC(O)CH₃; or R¹ and R² are joined together to form, together with the carbon to which they are attached, an oxo; R³ is selected from H and CH₃; R⁴ is H or absent; R⁵ is H or absent; n is selected from 0 and 1; m is selected from 0 and 1; and provided that: when - - - denotes no bond, both R⁴ and R⁵ are H; when - - - denotes a single bond, both R⁴ and R⁵ are absent; when n is 0, R¹ and R² are joined together to form an oxo, R³ is H, both R⁴ and R⁵ are H, - - - is no bond, and m is 1.

In some embodiments, provided herein is a method for inhibition of tumor cell growth, comprising administering an effective amount of a compound of Formula I

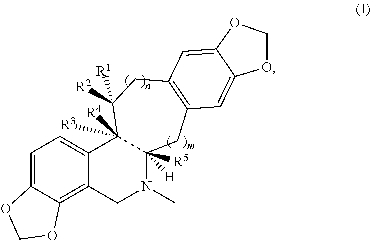

wherein: - - - denotes no bond or a single bond; R¹ is H; R² is selected from —OH and —OC(O)CH₃; or R¹ and R² are joined together to form, together with the carbon to which they are attached, an oxo; R³ is selected from H and CH₃; R⁴ is H or absent; R⁵ is H or absent; n is selected from 0 and 1; m is selected from 0 and 1; and provided that: when - - - denotes no bond, both R⁴ and R⁵ are H; when - - - denotes a single bond, both R⁴ and R⁵ are absent; when n is 0, R¹ and R² are joined together to form an oxo, R³ is H, both R⁴ and R⁵ are H, - - - is no bond, and m is 1.

In embodiments, provided herein is a pharmaceutical composition for inhibition of tumor cell growth, comprising an effective amount of a compound of Formula I

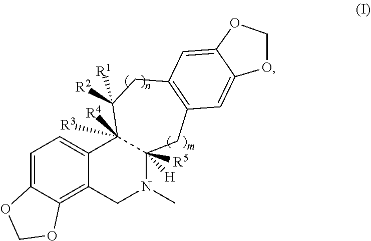

wherein: - - - denotes no bond or a single bond; R¹ is H; R² is selected from —OH and —OC(O)CH₃; or R¹ and R² are joined together to form, together with the carbon to which they are attached, an oxo; R³ is selected from H and CH₃; R⁴ is H or absent; R⁵ is H or absent; n is selected from 0 and 1; m is selected from 0 and 1; and provided that: when - - - denotes no bond, both R⁴ and R⁵ are H; when - - - denotes a single bond, both R⁴ and R⁵ are absent; when n is 0, R¹ and R² are joined together to form an oxo, R³ is H, both R⁴ and R⁵ are H, - - - is no bond, and m is 1.

In an embodiment, provided herein is a composition for inducing apoptosis in cancer cells, the composition comprising corynoline, acetylcorynoline, chelidonine, or protopine.

In some embodiments, provided herein is a method of treating cancer, the method comprising administering, to a person in need of such treatment, corynoline, acetylcorynoline, chelidonine, or protopine in an amount sufficient to induce apoptosis and inhibiting cell growth of cancer cells.

### BRIEF DESCRIPTION OF THE DRAWINGS

The present disclosure, in accordance with one or more various embodiments, is described in detail with reference to the following figures. The drawings are provided for purposes of illustration only and merely depict exemplary embodiments of the disclosure. These drawings are provided to facilitate the reader's understanding of the disclosure and should not be considered limiting of the breadth, scope, size, or applicability of the disclosure. It should be noted that for clarity and ease of illustration these drawings are not necessarily made to scale.

FIG. 1 shows an illustration of corynoline and acetylcorynoline extracted from Corydalis longicalcarata and an evaluation of their abilities in arresting cells in mitosis through both immunofluorescence microscopy and analysis of phosphorylated Histone H3 and DAPI analysis of polyploidy.

FIG. 2 shows IC₅₀ values of acetylcorynoline in RPE cell lines expressing oncogenic factors, and Rat1 A cell lines expressing oncogenic factors.

FIG. 3 shows IC₅₀ values of acetylcorynoline in human cancer cell lines.

FIG. 4 shows abnormal spindle polarization in mitosis following treatment with acetylcorynoline for 6 hours as seen by β-tubulin staining, with high numbers of cells arresting in prometaphase as determined by phospho-Histone H3 staining in immunofluorescence microscopy. Staining with DAPI 48 hours after treatment demonstrates polyploidy in the acetylcorynoline-treated conditions.

FIG. 5 shows multipolar spindle formation in mitosis following treatment with acetylcorynoline for 24 hours as indicated by β-tubulin staining. Staining with DAPI 48 hours after treatment demonstrates polyploidy in the acetylcorynoline-treated conditions.

FIG. 6 shows immunofluorescence images of cells stained with kinetochore-recognizing CREST antibody following 6 hours treatment with acetylcorynoline. Staining with DAPI 48 hours after treatment demonstrates polyploidy in the acetylcorynoline-treated conditions.

FIG. 7 shows immunofluorescence images of cells stained with kinetochore-recognizing CREST antibody following 24 hours treatment with acetylcorynoline. Staining with DAPI 48 hours after treatment demonstrates polyploidy in the acetylcorynoline-treated conditions.

FIG. 8 shows time lapse microscopy, beginning at prophase and tracking mitosis, for comparison of cells after treatment with acetylcorynoline and DMSO control group.

FIG. 9 shows IC₅₀ values of corynoline, acetylcorynoline, and taxol across multiple cell lines.

FIG. 10 shows IC₅₀ values of corynoline, acetylcorynoline, and taxol across multiple cell lines. FIG. 10A. shows IC₅₀ values of corynoline, acetylcorynoline, and taxol across RPE cells expressing oncogenes. FIG. 10B. shows IC₅₀ values of corynoline, acetylcorynoline, and taxol across Rat1 A cells expressing oncogenes. FIG. 10C shows IC₅₀ values of corynoline, acetylcorynoline, and taxol across human cancer cell lines. FIG. 10D shows IC₅₀ values of corynoline, acetylcorynoline, and taxol across additional cancer cell lines.

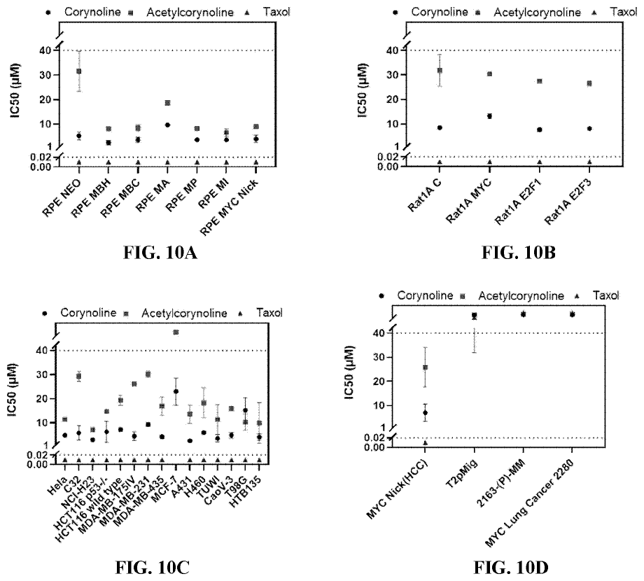

FIG. 11 shows IC₅₀ values of corynoline, acetylcorynoline, and taxol for RPE cell lines expressing oncogenes. FIG. 11A shows IC₅₀ values of corynoline. FIG. 11B shows IC₅₀ values of acetylcorynoline. FIG. 11C shows a comparison of IC₅₀ values of corynoline, acetylcorynoline, and taxol for the RPE cell lines. FIG. 11D shows a ratio of IC₅₀ values for acetylcorynoline to corynoline.

FIG. 12 shows IC₅₀ values of corynoline, acetylcorynoline, and taxol for Rat1A cell lines expressing oncogenes. FIG. 12A shows IC₅₀ values of corynoline. FIG. 12B shows IC₅₀ values of acetylcorynoline. FIG. 12C shows a comparison of IC₅₀ values of corynoline, acetylcorynoline, and taxol for the RPE cell lines. FIG. 12D shows a ratio of IC₅₀ values for acetylcorynoline to corynoline.

FIG. 13 shows IC₅₀ values of corynoline, acetylcorynoline, and taxol for multiple human cancer cell lines. FIG. 13A shows IC₅₀ values of corynoline. FIG. 13B shows IC₅₀ values of acetylcorynoline. FIG. 13C shows a comparison of IC₅₀ values of corynoline, acetylcorynoline, and taxol for the RPE cell lines. FIG. 13D shows a ratio of IC₅₀ values for acetylcorynoline to corynoline.

FIG. 14 shows bright field microscopy images of cells treated with DMSO, corynoline, acetylcorynoline, and vinblastine, in order to assay for mitosis by cell round-up. Staining with DAPI 48 hours after treatment demonstrates polyploidy in the corynoline and acetylcorynoline-treated conditions.

FIG. 15 shows that corynoline and acetylcorynoline elicit mitotic arrest and induce polyploidy in a concentration-dependent matter.

FIG. 16 shows bright field microscopy images of cells treated with DMSO, chelidonine, and protopine, in order to assay for mitosis by cell round-up. Staining with DAPI 48 hours after treatment demonstrates polyploidy in the chelidonine-treated conditions.

FIG. 17 shows multipolar spindle formation in mitosis following treatment with chelidonine for 24 hours as indicated by β-tubulin staining. Staining with DAPI 48 hours after treatment demonstrates polyploidy in the chelidonine-treated conditions.

### DETAILED DESCRIPTION OF EXEMPLARY EMBODIMENTS

As generally described herein, the present invention provides compounds to act as mitotic regulators. In certain embodiments, such compounds are envisioned to be useful as therapeutics agents for the treatment of cancers (e.g., a disorder described herein, a tumor, a cancer cell, etc.). The features and other details of the disclosure will now be more particularly described. Before further description of the present disclosure, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

### Definitions

The terms “a” and “an” as used herein mean “one or more” and include the plural unless the context is inappropriate.

A “subject” to which administration is contemplated includes, but is not limited to, humans (i.e., a male or female of any age group, e.g., a pediatric subject (e.g, infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

Disease, disorder, and condition are used interchangeably herein. In some embodiments, the disease is cancer. In some embodiments, the disease is caused by interference in the function of the mitotic spindle. In an embodiment, the disease is caused by apoptosis. In an embodiment, the disease is caused by mitotic catastrophe.

In general, the “effective amount” of a compound refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the age, health, and condition of the subject.

As used herein, the term “treat” or “treatment” is defined as the application or administration of a compound to a subject, e.g., a patient, or application or administration of the compound to an isolated tissue or cell, e.g., cell line, from a subject, e.g., a patient, who has a disease such as cancer.

As used herein, an amount of a compound effective to treat a disorder, or a “therapeutically effective amount” refers to an amount of the compound which is effective, upon single or multiple dose administration to a subject, in treating a cell, or in curing, alleviating, relieving or improving a subject with a disorder beyond that expected in the absence of such treatment.

The term “acetylcorynoline,” as used herein, refers to a compound of the following structure:

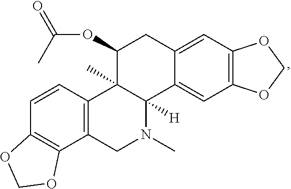 [SMILES: CC(=O)O[C@H]1Cc2cc3c(cc2[C@H]2N(C)Cc4c(ccc5c4OCO5)[C@@]12C)OCO3]

or a pharmaceutically acceptable salt, thereof. The CAS registry number for acetylcorynoline is 18797-80-3. Other names for acetylcorynoline include, but are not limited to: ACETYLCORYNOLINE(P), (5bR,6S,12bR)-5b,6,7,12b,13,14-Hexahydro-5b,13-dimethyl-[1,3]benzodioxolo[5,6-c]-1,3-dioxolo[4,5-i]phenanthridin-6-ol 6-acetate, corynoline acetate, O-Acetylcorynoline, [1,3]Benzodioxolo[5,6-c]-1,3-dioxolo[4,5-i]phenanthridin-6-ol,5b,6,7,12b,13,14-hexahydro-5b,13-dimethyl-, and 6-acetate, (5bR,6S,12bR)-.

The term “corynoline,” as used herein, refers to a compound of the following structure:

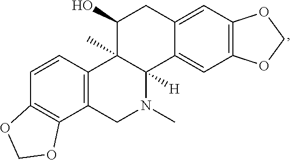 [SMILES: CN1Cc2c(ccc3c2OCO3)[C@@]2(C)[C@@H](O)Cc3cc4c(cc3[C@@H]12)OCO4]

or a pharmaceutically acceptable salt, thereof. The CAS registry number for corynoline is 18797-79-0. Other names for corynoline include, but are not limited to: [1,3]Benzodioxolo[5,6-c]-1,3-dioxolo[4,5-i]phenanthridin-6-o1,5b,6,7,12b, 13,14-hexahydro-5b,13-, dimethyl-, (5bR,6S,12bR)-, (5bR)-5bα,13-Dimethyl-5bα,6,7,12bα,13,14-hexahydro[1,3]benzodioxolo[5,6-c]-1,3-dioxolo[4,5-i]phenanthridine-6β-ol, 13-Methylchelidonan-11β-ol, and CORYNOLINE(P).

The term “chelidonine,” as used herein, refers to a compound of the following structure:

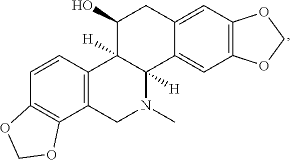 [SMILES: CN1Cc2c(ccc3c2OCO3)[C@@H]2[C@H]1c1cc3c(cc1C[C@@H]2O)OCO3]

or a pharmaceutically acceptable salt, thereof. The CAS registry number for chelidonine is 476-32-4. Other names for chelidonine include, but not limited to: 5bR,6S,7,12bS,13,14-hexahydro-13-methyl-[1,3]benzodioxolo[5,6-c]-1,3-dioxolo[4,5-i]phenanthridin-6-ol, chelidoniny, helidonine, khelidonin, Stylophorin, Stylophorine, Stylophoron, And Chelidonin.

The term “protopine,” as used herein, refers to a compound of the following structure:

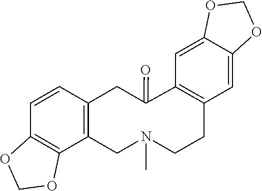 [SMILES: CN1CCc2cc3c(cc2C(=O)Cc2ccc4c(c2C1)OCO4)OCO3]

or a pharmaceutically acceptable salt, thereof. The CAS registry number for protopine is 6164-47-2. Other names for protopine include, but not limited to: 7-Methyl-6,8,9,16-tetrahydrobis[1,3]benzodioxolo[4,5-c:5′,6′-g]azecin-15(7H)-one, 4,6,7,14-tetrahydro-5-methyl-bis(1,3)benzodioxolo(4,5-c-5′,6′-g)azecin-13(5h)-one, Corydinine, Fumarine, Biflorine, and Macleyine.

The term “oxo” as used herein refers to the radical=O.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like. See, e.g., Berge, et al., J. Pharm. Sci. (1977) 66 (1): 1-79.

The term “prodrug” is intended to encompass compounds that, under physiological conditions, are converted into the therapeutically active agents of the present invention. A common method for making a prodrug is to include selected moieties that are hydrolyzed under physiological conditions to reveal the desired molecule. In other embodiments, the prodrug is converted by an enzymatic activity of the subject.

“Solvate” refers to forms of the compound that are associated with a solvent or water (also referred to as “hydrate”), usually by a solvolysis reaction. This physical association includes hydrogen bonding. Conventional solvents include water, ethanol, acetic acid, and the like. The compounds of the invention may be prepared e.g., in crystalline form and may be solvated or hydrated. Suitable solvates include pharmaceutically acceptable solvates, such as hydrates, and further include both stoichiometric solvates and non-stoichiometric solvates. In certain instances the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

“Stereoisomers”: It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

“Tautomers” refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest.

A “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”).

The term “mitotic regulator” as used herein refers to compounds, for example, that act to regulate mitotic cell cycle. Mitotic regulators can be used to treat disorders such as cancer, including but not limited to: bladder cancer, breast cancer, cervical cancer, glioblastoma, head and neck cancer, lung cancer, liver cancer, melanoma, ovarian cancer, pancreatic cancer, prostate cancer, sarcoma, renal cancer, colorectal cancer, gastric cancer, neuroblastoma, squamous cell carcinoma, or acute myeloid leukemia (AML). In certain embodiments, disorders such as cancer that mitotic regulators can treat is non-Hodgkin's lymphoma, such as a B-cell lymphoma or a T-cell lymphoma. In certain embodiments, the non-Hodgkin's lymphoma is a B-cell lymphoma, such as a diffuse large B-cell lymphoma, primary mediastinal B-cell lymphoma, follicular lymphoma, small lymphocytic lymphoma, mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, hairy cell leukemia, or primary central nervous system (CNS) lymphoma. In certain other embodiments, the non-Hodgkin's lymphoma is a T-cell lymphoma, such as a precursor T-lymphoblastic lymphoma, peripheral T-cell lymphoma, cutaneous T-cell lymphoma, angioimmunoblastic T-cell lymphoma, extranodal natural killer/T-cell lymphoma, enteropathy type T-cell lymphoma, subcutaneous panniculitis-like T-cell lymphoma, anaplastic large cell lymphoma, or peripheral T-cell lymphoma.

In some embodiments, the mitotic regulators are used for treatment of cancers with high proliferative index. A high proliferation rate is determined by methods known in the art, such as, for example and without limitation, Ki67 staining of tissue or cells.

In certain embodiments, the mitotic regulators are used for treatment of MYC-expressing cancers. MYC is frequently dysregulated, for example, in multiple myeloma. MYC is frequently activated aberrantly in colon cancer. Additionally, MYC is amplified in about 14% of cancers, including, without limitation, ovarian cancer, breast cancer, squamous cell lung cancer, small cell lung cancer, and neuroblastoma (Kalkat et al., Genes (2017) 8 (6):151)). In certain embodiments, the cancer to be treated is breast cancer and/or lung cancer. In certain embodiments, the cancer to be treated is breast cancer In certain embodiments, the cancer to be treated is lung cancer.

In certain embodiments, the mitotic regulators are used for treatment of cancers with aberrant BCL-2 expression and/or activation.

In certain embodiments, the mitotic regulators are used for treatment of proliferative disorders such as, for example and without limitation, Castelman's disease, familial adenomatous polyposis, nevi, primary sclerosing cholangitis, lesions from Human Papillomavirus Infections, and myeloproliferative disorders.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

### Compounds

In an aspect, provided herein are compounds of Formula I

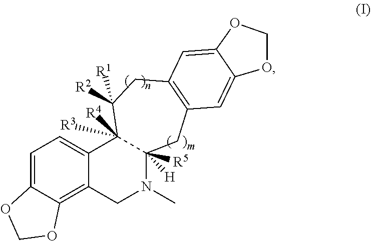

wherein: - - - denotes no bond or a single bond; R¹ is H; R² is selected from —OH and —OC(O)CH₃; or R¹ and R² are joined together to form, together with the carbon to which they are attached, an oxo; R³ is selected from H and CH₃; R⁴ is H or absent; R⁵ is H or absent; n is selected from 0 and 1; m is selected from 0 and 1; and provided that: when - - - denotes no bond, both R⁴ and R⁵ are H; when - - - denotes a single bond, both R⁴ and R⁵ are absent; when n is 0, R¹ and R² are joined together to form an oxo, R³ is H, both R⁴ and R⁵ are H, - - - is no bond, and m is 1.

In some embodiments, - - - is a single bond.

In some embodiments, - - - is no bond.

In some embodiments, R² is —OH.

In some embodiments, R² is —OC(O)CH₃.

In some embodiments, R¹ and R² are joined together to form, together with the carbon to which they are attached, an oxo.

In some embodiments, R³ is H.

In some embodiments, R³ is CH₃.

In some embodiments, R⁴ is H.

In some embodiments, R⁴ is absent.

In some embodiments, R⁵ is H.

In some embodiments, R⁵ is absent.

In some embodiments, n is 0.

In some embodiments, n is 1.

In some embodiments, m is 0.

In some embodiments, m is 1.

In some embodiments, R¹ is H, R² is —OH, R³ is CH₃, - - - is a single bond, n is 1, and m is 0.

In some embodiments, R¹ is H, R² is —OC(O)CH₃, R³ is CH₃, - - - is a single bond, n is 1, and m is 0.

In some embodiments, R¹ is H, R² is —OH, R³ is H, - - - is a single bond, n is 1, and m is 0.

In some embodiments, n is 0, R¹ and R² are joined together to form an oxo, R³ is H, both R⁴ and R¹ are H, - - - is no bond, and m is 1.

In some aspects, the compound is

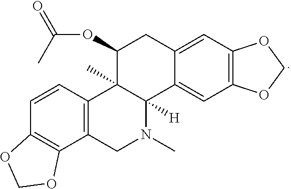 [SMILES: CC(=O)O[C@H]1Cc2cc3c(cc2[C@H]2N(C)Cc4c(ccc5c4OCO5)[C@@]12C)OCO3]

In some other aspects, the compound is

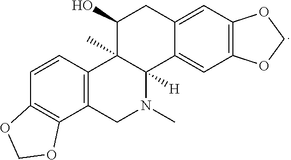 [SMILES: CN1Cc2c(ccc3c2OCO3)[C@@]2(C)[C@@H](O)Cc3cc4c(cc3[C@@H]12)OCO4]

In some other aspects, the compound is

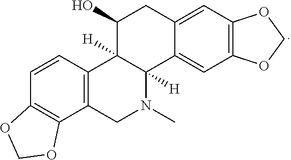 [SMILES: CN1Cc2c(ccc3c2OCO3)[C@@H]2[C@H]1c1cc3c(cc1C[C@@H]2O)OCO3]

In some other aspects, the compound is

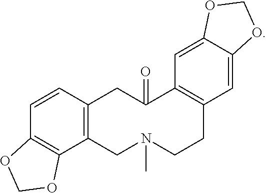 [SMILES: CN1CCc2cc3c(cc2C(=O)Cc2ccc4c(c2C1)OCO4)OCO3]

In an embodiment, the compound is isolated and purified from the rhizomes of Corydalis longicalcarata. In an embodiment, the compound is a mitotic inhibitor.

In embodiments, the compound promotes anti-mitotic activities through pleiotropic effects on cell division. In some embodiments, pleiotropic effects include comprising cell division, prevention of chromosome congression, compromise of spindle checkpoint response, and blockage of cytokinesis.

In an aspect, provided herein is a pharmaceutical composition comprising a compound described herein (e.g., a compound of Formula I) or a pharmaceutically acceptable salt thereof, and a pharmaceutically acceptable excipient. In certain embodiments, the compound of the present invention is provided in an effective amount in the pharmaceutical composition. In certain embodiments, the compound of the present invention is provided in a therapeutically effective amount. In certain embodiments, the compound of the present invention is provided in a prophylactically effective amount.

In an aspect, provided herein is a pharmaceutically acceptable salt of a compound described herein (e.g., a compound of Formula I).

In certain embodiments, the compound is administered orally, subcutaneously, intravenously, or intramuscularly. In certain embodiments, the compound is administered orally. In certain embodiments, the compound is administered chronically. In certain embodiments, the compound is administered continuously, e.g., by continuous intravenous infusion.

### Methods of Use and Treatment

Compounds of the present invention as described herein, act, in certain embodiments, as mitotic regulators, e.g., effecting mitosis in either a positive or negative manner. As modulators of mitosis, such compounds are expected to treat cancers.

In some aspects, compounds of the disclosure suppress tumor function as a mitotic regulator.

In an aspect, described herein is a method of treating cancer in a subject in need thereof, comprising administering to the subject in need thereof an effective amount of a compound described herein or a pharmaceutically acceptable salt thereof.

Compounds described herein can act as mitotic regulators in the treatment of a disease or disorder in a patient in need thereof. The disease or disorder, for example, can be a cancer. In other aspects, compounds described herein can treat a tumor, e.g., a solid tumor. In other aspects, compounds described herein can treat a tumor, e.g., a liquid tumor.

Provided herein is a method of treating cancer in a subject thereof, the method comprising administering to the subject in need thereof a therapeutically effective amount of a compound of Formula I

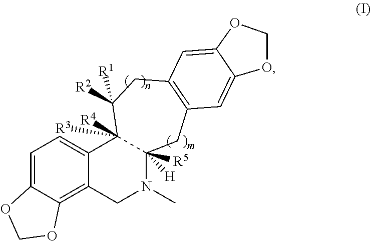

wherein: - - - denotes no bond or a single bond; R¹ is H; R² is selected from —OH and —OC(O)CH₃; or R¹ and R² are joined together to form, together with the carbon to which they are attached, an oxo; R³ is selected from H and CH₃; R⁴ is H or absent; R⁵ is H or absent; n is selected from 0 and 1; m is selected from 0 and 1; and provided that: when - - - denotes no bond, both R⁴ and R⁵ are H; when - - - denotes a single bond, both R⁴ and R⁵ are absent; when n is 0, R¹ and R² are joined together to form an oxo, R³ is H, both R⁴ and R⁵ are H, - - - is no bond, and m is 1.

In some embodiments, the cancer is selected from the group consisting of ovarian cancer, lung cancer, gastric cancer, breast cancer, hepatic cancer, pancreatic cancer, skin cancer, malignant melanoma, head and neck cancer, sarcoma, bile duct cancer, cancer of the urinary bladder, kidney cancer, colon cancer, small bowel cancer, testicular embryonal 5 carcinoma, placental choriocarcinoma, cervical cancer, testicular cancer, uterine cancer, a germ cell tumor and the metastatic forms thereof.

Also provided herein is a method of inducing tumor cell apoptosis in a subject, the method comprising: administering to the subject in need thereof a therapeutically effective amount of a compound of Formula I

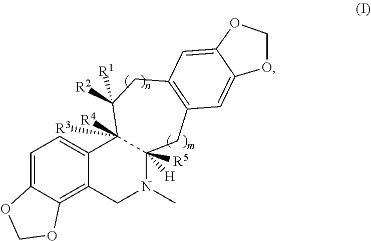

wherein: - - - denotes no bond or a single bond; R¹ is H; R² is selected from —OH and —OC(O)CH₃; or R¹ and R² are joined together to form, together with the carbon to which they are attached, an oxo; R³ is selected from H and CH₃; R⁴ is H or absent; R⁵ is H or absent; n is selected from 0 and 1; m is selected from 0 and 1; and provided that: when - - - denotes no bond, both R⁴ and R⁵ are H; when - - - denotes a single bond, both R⁴ and R⁵ are absent; when n is 0, R¹ and R² are joined together to form an oxo, R³ is H, both R⁴ and R⁵ are H, - - - is no bond, and m is 1.

In an embodiment, provided herein is a method of arresting cell mitosis in a cell in a subject, the method comprising: administering to the subject in need thereof a therapeutically effective amount of a compound of Formula I

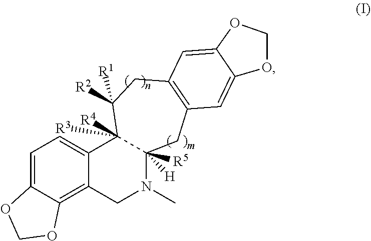

wherein: - - - denotes no bond or a single bond; R¹ is H; R² is selected from —OH and —OC(O)CH₃; or R¹ and R² are joined together to form, together with the carbon to which they are attached, an oxo; R³ is selected from H and CH₃; R⁴ is H or absent; R⁵ is H or absent; n is selected from 0 and 1; m is selected from 0 and 1; and provided that: when - - - denotes no bond, both R⁴ and R⁵ are H; when - - - denotes a single bond, both R⁴ and R⁵ are absent; when n is 0, R¹ and R² are joined together to form an oxo, R³ is H, both R⁴ and R⁵ are H, - - - is no bond, and m is 1.

### Disruption of Mitosis

The cell cycle is the process whereby cells grow and divide. The cell cycle is composed of two phases: interphase (which consists of G1, S, and G2 phases), and mitosis. When a cell has duplicated its chromosomes in S phase and receives the signal to divide, it exits G2 and enters mitosis.

Mitosis is the part of the cell cycle during which replicated chromosomes are separated into two new nuclei, and two daughter cells. It is characterized by its five stages: prophase, prometaphase, metaphase, anaphase, and telophase. During mitosis, the duplicated chromosomes are first condensed, followed by attachment by kinetochore microtubules, also known as spindle fibers. The spindle fibers come from the centrosomes that replicated themselves during the G1 stage of interphase. During prometaphase, spindle fibers will be projected from the centrosomes and connect with the aligned chromosomes. Microtubules, which contain a- and β-tubulin, play an essential role in these early processes of mitosis, such as regulation of cell movement, cytoplasmic transport, and chromosome alignment. However, when microtubules are disrupted, monopolar and multipolar spindles will be produced, causing mitotic catastrophe. Prolonged mitosis, or mitotic arrest, results in cellular apoptosis.

Inhibitors of microtubule activity known in the art, e.g., taxol and nocodazole, are amongst the earliest chemotherapeutic agents for the treatment of cancer. Taxol (paclitaxel) is a microtubule inhibitor that stabilizes the microtubules and prevents disassembly, resulting in mitotic arrest and cellular apoptosis. Nocodazole, a microtubule inhibitor that disrupts polymerization of microtubules and arrests treated cells in mitosis, is also frequently used in biological research, e.g., in academic and industry biology laboratory benchwork. The inhibitors of the present invention, corynoline or acetylcorynoline, are also contemplated for use in biological research, e.g., in academic and industry biology laboratory benchwork.

In some embodiments, compounds of the present invention can induce centrosome duplication in mitosis and the formation of multipolar spindles.

In another embodiment, provided herein is a method of modulating mitotic index of a cell in a subject in need thereof, the method comprising administering to the subject in need thereof a therapeutically effective amount of a compound of Formula I

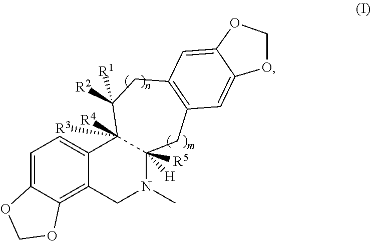

wherein: - - - denotes no bond or a single bond; R¹ is H; R² is selected from —OH and —OC(O)CH₃; or R¹ and R² are joined together to form, together with the carbon to which they are attached, an oxo; R³ is selected from H and CH₃; R⁴ is H or absent; R⁵ is H or absent; n is selected from 0 and 1; m is selected from 0 and 1; and provided that: when - - - denotes no bond, both R⁴ and R⁵ are H; when - - - denotes a single bond, both R⁴ and R⁵ are absent; when n is 0, R¹ and R² are joined together to form an oxo, R³ is H, both R⁴ and R⁵ are H, - - - is no bond, and m is 1.

In some embodiments, provided herein is a method of modulating a mitotic regulator, comprising administering an effective amount of a compound of Formula I

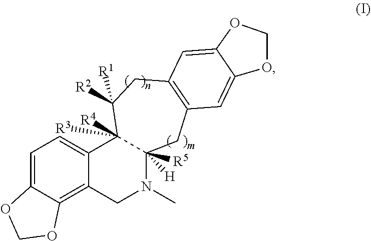

wherein: - - - denotes no bond or a single bond; R¹ is H; R² is selected from —OH and —OC(O)CH₃; or R¹ and R² are joined together to form, together with the carbon to which they are attached, an oxo; R³ is selected from H and CH₃; R⁴ is H or absent; R⁵ is H or absent; n is selected from 0 and 1; m is selected from 0 and 1; and provided that: when - - - denotes no bond, both R⁴ and R⁵ are H; when - - - denotes a single bond, both R⁴ and R⁵ are absent; when n is 0, R¹ and R² are joined together to form an oxo, R³ is H, both R⁴ and R⁵ are H, - - - is no bond, and m is 1.

In some embodiments, provided herein is a method for inhibition of tumor cell growth, comprising administering an effective amount of a compound of Formula I

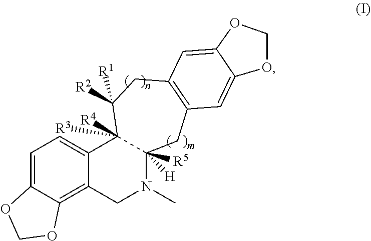

wherein: - - - denotes no bond or a single bond; R¹ is H; R² is selected from —OH and —OC(O)CH₃; or R¹ and R² are joined together to form, together with the carbon to which they are attached, an oxo; R³ is selected from H and CH₃; R⁴ is H or absent; R⁵ is H or absent; n is selected from 0 and 1; m is selected from 0 and 1; and provided that: when - - - denotes no bond, both R⁴ and R⁵ are H; when - - - denotes a single bond, both R⁴ and R⁵ are absent; when n is 0, R¹ and R² are joined together to form an oxo, R³ is H, both R⁴ and R¹ are H, - - - is no bond, and m is 1.

In some embodiments, - - - is a single bond. In some embodiments, - - - is no bond. In some embodiments, R² is —OH. In some embodiments, R² is —OC(O)CH₃. In some embodiments, R¹ and R² are joined together to form, together with the carbon to which they are attached, an oxo. In some embodiments, R³ is H. In some embodiments, R³ is CH₃. In some embodiments, R⁴ is H. In some embodiments, R⁴ is absent. In some embodiments, R⁵ is H. In some embodiments, R⁵ is absent. In some embodiments, n is 0. In some embodiments, n is 1. In some embodiments, m is 0. In some embodiments, m is 1. In some embodiments, R¹ is H, R² is —OH, R³ is CH₃, - - - is a single bond, n is 1, and m is 0. In some embodiments, R¹ is H, R² is —OC(O)CH₃, R³ is CH₃, - - - is a single bond, n is 1, and m is 0. In some embodiments, R¹ is H, R² is —OH, R³ is H, - - - is a single bond, n is 1, and m is 0. In some embodiments, n is 0, R¹ and R² are joined together to form an oxo, R³ is H, both R⁴ and R⁵ are H, - - - is no bond, and m is 1.

In an embodiment, the compound is isolated and purified from the rhizomes of Corydalis longicalcarata. In an embodiment, the compound is a mitotic inhibitor.

In embodiments, the compound promotes anti-mitotic activities through pleiotropic effects on cell division. In some embodiments, pleiotropic effects comprise defective cell division, prevention of chromosome congression, compromise of spindle checkpoint response, and blockage of cytokinesis. In some embodiments, the compound promotes formation of multinucleated cells. Without wishing to be bound by theory, it is hypothesized that administration of a compound of the present disclosure results in i.) formation of supernumerary centrosomes in early mitosis that form multipolar mitotic spindles, leading to prolonged mitotic arrest and subsequent apoptosis, ii.) blockade of cytokinesis, which results in accumulation of multinucleated and polyploid cells that either undergo apoptosis following mitotic catastrophe or enter a senescent state, inhibiting tumor growth and/or progression.

In embodiments, the method is a chemotherapy. In some embodiments, the administering is carried out in combination with another cancer therapy.

In some embodiments, provided herein is a method of treating cancer, the method comprising administering, to a subject in need of such treatment, corynoline, acetylcorynoline, chelidonine, or protopine in an amount sufficient to induce apoptosis and/or inhibit cell growth of cancer cells, i.e., a therapeutically effective amount.

### Pharmaceutical Compositions

In an embodiment, provided herein is a pharmaceutical composition for inhibition of tumor cell growth, comprising an effective amount of a compound of Formula I

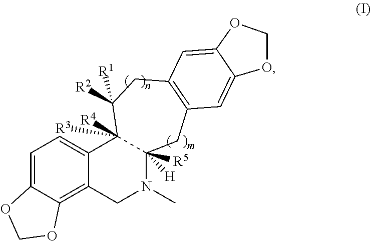

wherein: - - - denotes no bond or a single bond; R¹ is H; R² is selected from —OH and —OC(O)CH₃; or R¹ and R² are joined together to form, together with the carbon to which they are attached, an oxo; R³ is selected from H and CH₃; R⁴ is H or absent; R⁵ is H or absent; n is selected from 0 and 1; m is selected from 0 and 1; and provided that: when - - - denotes no bond, both R⁴ and R⁵ are H; when - - - denotes a single bond, both R⁴ and R⁵ are absent; when n is 0, R¹ and R² are joined together to form an oxo, R³ is H, both R⁴ and R¹ are H, - - - is no bond, and m is 1.

In some embodiments, - - - is a single bond. In some embodiments, - - - is no bond. In some embodiments, R² is —OH. In some embodiments, R² is —OC(O)CH₃. In some embodiments, R¹ and R² are joined together to form, together with the carbon to which they are attached, an oxo. In some embodiments, R³ is H. In some embodiments, R³ is CH₃. In some embodiments, R⁴ is H. In some embodiments, R⁴ is absent. In some embodiments, R⁵ is H. In some embodiments, R⁵ is absent. In some embodiments, n is 0. In some embodiments, n is 1. In some embodiments, m is 0. In some embodiments, m is 1. In some embodiments, R¹ is H, R² is —OH, R³ is CH₃, - - - is a single bond, n is 1, and m is 0. In some embodiments, R¹ is H, R² is —OC(O)CH₃, R³ is CH₃, - - - is a single bond, n is 1, and m is 0. In some embodiments, R¹ is H, R² is —OH, R³ is H, - - - is a single bond, n is 1, and m is 0. In some embodiments, n is 0, R¹ and R² are joined together to form an oxo, R³ is H, both R⁴ and R⁵ are H, - - - is no bond, and m is 1.

In an embodiment, the tumor cell is selected from the group consisting of hepatoma cell, an esophageal cancer cell, a cervical adenocarcinoma cell, a pancreatic cancer cell, and a leukemic cell. In another embodiment, the compound is isolated and purified from the rhizomes of Corydalis longicalcarata. In some embodiments, the compound is a mitotic inhibitor. In some embodiments, the compound promotes anti-mitotic activities through pleiotropic effects on cell division. In an embodiment, the pleiotropic effects include compromise of cell division, prevention of chromosome congression, compromise of spindle checkpoint response, and blockage of cytokinesis.

Also provided herein is a composition for inducing apoptosis in cancer cells, the composition comprising corynoline, acetylcorynoline, chelidonine, or protopine.

### Combination Therapy

Another aspect of the invention provides for combination therapy. A compound that can act as a mitotic regulator described herein can be used in combination with additional therapeutic agents to treat cancer.

Exemplary therapeutic agents that may be used as part of a combination therapy in treating cancer, include, for example, radiation, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma (IFN-γ), colony stimulating factor-1, colony stimulating factor-2, denileukin diftitox, interleukin-2, luteinizing hormone releasing factor and variations of the aforementioned agents that may exhibit differential binding to its cognate receptor, or increased or decreased serum half-life.

Additionally, immune checkpoint inhibitors may be used in combination with the compounds of the present disclosure in the treatment of cancer. Exemplary immune checkpoint inhibitors include agents that inhibit one or more of (i) cytotoxic T lymphocyte-associated antigen 4 (CTLA4), (ii) programmed cell death protein 1 (PD1), (iii) PDL1, (iv) LAG3, (v) B7-H3, (vi) B7-H4, and (vii) TIM3. For example, the CTLA4 inhibitor ipilimumab, or the PD1/PD-L1 inhibitor pembrolizumab, may be used in combination with the compounds of the present disclosure.

Targeted protein inhibitors such as, without limitation, signal transduction inhibitors (e.g., PI3K inhibitors, EGFR inhibitors, etc.), angiogenesis inhibitors (e.g., VEGF inhibitors), and monoclonal antibodies (e.g., antibody-drug conjugates), are also contemplated for use in combination with the compounds of the present disclosure. In certain embodiments, a MYC-inhibitor is used in combination with a compound of the present disclosure.

In some embodiments, the compounds of the present disclosure are combined with surgical interventions, where abnormal tissue (e.g., a tumor) is surgically removed from the subject in need thereof. In some embodiments, the tumor is cut from the subject's body using scalpels or other sharp tools to excise the tumor and/or surrounding tissue. In some embodiments, lasers can be used to cut abnormal tissue (e.g., a tumor). The surgical intervention may involve open surgery or minimally invasive surgery depending on the tumor type being removed. In some embodiments, the surgical intervention can be used to remove the entire tumor, to debulk a tumor, or to reduce cancer symptoms. The compounds of the present disclosure may be administered prior to surgery. The compounds of the present disclosure may be administered concurrently with surgery. The compounds of the present disclosure may be administered subsequent to surgery.

The amount of mitotic regulator and additional therapeutic agent and the relative timing of administration may be selected in order to achieve a desired combined therapeutic effect. For example, when administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. Further, for example, a mitotic regulator may be administered during a time when the additional therapeutic agent(s) exerts its prophylactic or therapeutic effect, or vice versa.

### Pharmaceutical Compositions

In one aspect, provided herein is a pharmaceutical composition comprising a compound described herein (e.g., a compound of Formula I) or a pharmaceutically acceptable salt thereof, and a pharmaceutically acceptable excipient. In certain embodiments, the compound of the present invention is provided in an effective amount in the pharmaceutical composition. In certain embodiments, the compound of the present invention is provided in a therapeutically effective amount. In certain embodiments, the compound of the present invention is provided in a prophylactically effective amount.

In certain embodiments, the pharmaceutical composition comprises an effective amount of the active ingredient. In certain embodiments, the pharmaceutical composition comprises a therapeutically effective amount of the active ingredient. In certain embodiments, the pharmaceutical composition comprises a prophylactically effective amount of the active ingredient.

The pharmaceutical compositions provided herein can be administered by a variety of routes including, but not limited to, oral (enteral) administration, parenteral (by injection) administration, rectal administration, transdermal administration, intradermal administration, intrathecal administration, subcutaneous (SC) administration, intravenous (IV) administration, intramuscular (IM) administration, and intranasal administration.

Generally, the compounds provided herein are administered in an effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The compounds provided herein will be administered to a subject at risk for developing the condition, typically on the advice and under the supervision of a physician, at the dosage levels described above. Subjects at risk for developing a particular condition generally include those that have a family history of the condition, or those who have been identified by genetic testing or screening to be particularly susceptible to developing the condition.

The pharmaceutical compositions of the present invention may be further delivered using a variety of dosing methods. For example, in certain embodiments, the pharmaceutical composition may be given as a bolus, e.g., in order to raise the concentration of the compound in the blood to an effective level. The placement of the bolus dose depends on the systemic levels of the active ingredient desired throughout the body, e.g., an intramuscular or subcutaneous bolus dose allows a slow release of the active ingredient, while a bolus delivered directly to the veins (e.g., through an IV drip) allows a much faster delivery which quickly raises the concentration of the active ingredient in the blood to an effective level. In other embodiments, the pharmaceutical composition may be administered as a continuous infusion, e.g., by IV drip, to provide maintenance of a steady-state concentration of the active ingredient in the subject's body. Furthermore, in still yet other embodiments, the pharmaceutical composition may be administered as first as a bolus dose, followed by continuous infusion.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or preferably from about 1 to about 40% by weight) with the remainder being various vehicles or excipients and processing aids helpful for forming the desired dosing form.

With oral dosing, one to five and especially two to four and typically three oral doses per day are representative regimens. Using these dosing patterns, each dose provides from about 0.01 to about 20 mg/kg of the compound provided herein, with preferred doses each providing from about 0.1 to about 10 mg/kg, and especially about 1 to about 5 mg/kg.

Transdermal doses are generally selected to provide similar or lower blood levels than are achieved using injection doses, generally in an amount ranging from about 0.01 to about 20% by weight, preferably from about 0.1 to about 20% by weight, preferably from about 0.1 to about 10% by weight, and more preferably from about 0.5 to about 15% by weight.

Injection dose levels range from about 0.1 mg/kg/hour to at least 20 mg/kg/hour, all for from about 1 to about 120 hours and especially 24 to 96 hours. A preloading bolus of from about 0.1 mg/kg to about 10 mg/kg or more may also be administered to achieve adequate steady state levels. The maximum total dose is not expected to exceed about 5 g/day for a 40 to 80 kg human patient.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable excipients known in the art. As before, the active compound in such compositions is typically a minor component, often being from about 0.05 to 10% by weight with the remainder being the injectable excipient and the like.

Transdermal compositions are typically formulated as a topical ointment or cream containing the active ingredient(s). When formulated as an ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or formulation. All such known transdermal formulations and ingredients are included within the scope provided herein.

The compounds provided herein can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

The compounds of the present invention can also be administered in sustained release forms or from sustained release drug delivery systems. A description of representative sustained release materials can be found in Remington's Pharmaceutical Sciences.

The present invention also relates to the pharmaceutically acceptable acid addition salt of a compound of the present invention. The acid which may be used to prepare the pharmaceutically acceptable salt is that which forms a non-toxic acid addition salt, i.e., a salt containing pharmacologically acceptable anions such as the hydrochloride, hydroiodide, hydrobromide, nitrate, sulfate, bisulfate, phosphate, acetate, lactate, citrate, tartrate, succinate, maleate, fumarate, benzoate, para-toluenesulfonate, and the like.

In another aspect, the invention provides a pharmaceutical composition comprising a compound of the present invention and a pharmaceutically acceptable excipient, e.g., a composition suitable for injection, such as for intravenous (IV) administration.

Pharmaceutically acceptable excipients include any and all diluents or other liquid vehicles, dispersion or suspension aids, surface active agents, isotonic agents, preservatives, lubricants and the like, as suited to the particular dosage form desired, e.g., injection. General considerations in the formulation and/or manufacture of pharmaceutical compositions agents can be found, for example, in Remington's Pharmaceutical Sciences, Sixteenth Edition, E. W. Martin (Mack Publishing Co., Easton, Pa., 1980), and Remington: The Science and Practice of Pharmacy, 21^{st} Edition (Lippincott Williams & Wilkins, 2005).

For example, injectable preparations, such as sterile injectable aqueous suspensions, can be formulated according to the known art using suitable dispersing or wetting agents and suspending agents. Exemplary excipients that can be employed include, but are not limited to, water, sterile saline or phosphate-buffered saline, or Ringer's solution.

The injectable composition can be sterilized, for example, by filtration through a bacterial-retaining filter, or by incorporating sterilizing agents in the form of sterile solid compositions which can be dissolved or dispersed in sterile water or other sterile injectable medium prior to use.

Generally, the compounds provided herein are administered in an effective amount. The amount of the compound actually administered will typically be determined by a physician, in the light of the relevant circumstances, including the condition to be treated, the chosen route of administration, the actual compound administered, the age, weight, response of the individual patient, the severity of the patient's symptoms, and the like.

The compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include pre-filled, pre-measured ampules or syringes of the liquid compositions. In such compositions, the compound is usually a minor component (from about 0.1% to about 50% by weight or preferably from about 1% to about 40% by weight) with the remainder being various vehicles or carriers and processing aids helpful for forming the desired dosing form.

The compounds provided herein can be administered as the sole active agent, or they can be administered in combination with other active agents. In one aspect, the present invention provides a combination of a compound of the present invention and another pharmacologically active agent. Administration in combination can proceed by any technique apparent to those of skill in the art including, for example, separate, sequential, concurrent, and alternating administration.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation. General considerations in the formulation and/or manufacture of pharmaceutical compositions can be found, for example, in Remington: The Science and Practice of Pharmacy 21^{st} ed., Lippincott Williams & Wilkins, 2005.

In one aspect, provided is a kit comprising a composition comprising a compound of Formula I.

### Examples

In order that the invention described herein may be more fully understood, the following examples are set forth. The synthetic and biological examples described in this application are offered to illustrate the compounds, pharmaceutical compositions, and methods provided herein and are not to be construed in any way as limiting their scope.

### Materials and Methods

### Plant Materials:

The rhizomes of Corydalis longicalcarata were collected in April 2018 from Longchi National Forest Park, located in the northwest of Dujiangyan, Sichuan Province, China. This original plant was authenticated as Corydalis longicalcarata by professor Linfang Huang, Institute of Medicinal Plant Development, Peking Union Medical College & Chinese Academy of Medical Sciences, Beijing, China. A voucher specimen was assigned a number of MBICR-0728 and deposited at the Herbarium of the J. Michael Bishop Institute of Cancer Research (MBICR).

### Chemicals and Reagents:

The reference standards for corynoline and acetylcorynoline were purchased from Chengdu Must Biotechnology Co. Ltd. (Sichuan, China). The purity of these two natural compounds were higher than 98% as validated by analytic HPLC in-house. Silica gel (200-300 mesh) and all solvents used for extraction and isolation were from Chengdu Kelong Chemical Co., Ltd (Sichuan, China). Preparative TLC plates (silica gel, 200 mm×200 mm×1mm) were obtained from Yantai Xinnuo Co. Ltd. (Shangdong, China). All HPLC grade solvents were from Fisher Scientific (Fisher Scientific, USA) and used without further purification. Taxol and Vineblastine were purchased from Sigma. Deionized water was purified using a Milli-Q system (Millipore, Bedford, MA, USA).

### Cell Culture and Treatment:

Retinal pigment epithelial cells transformed by ectopic MYC and Bcl-2 expression (RPE-MBC cells) have been described previously (e.g., Goga et al., 2007, Nat. Med., 13 (7):820-827) and were cultured in DMEM supplemented with 5% fetal bovine serum in a humidified incubator that was maintained at 5% CO2. For screening for bioactive fractions, cells were cultured in 96-well plates, and exposed to crude extracts, partially purified fractions or pure compounds at the concentrations indicated in the figure legends. Exposure was for 24 or 48 hours before analysis of cells for either an arrest in mitosis using an inverted microscope or for a change in DNA content after 4′,6′-diamidino-2-phenylindole (DAPI) staining by fluorescence microscopy.

The RPE series were derived from human retinal pigment epithelium and then stably transfected with a hTERT expression construct for immortalization. The resulting cell line was then engineered to overexpress the MYC and BCL2 oncogenes, generating the RPE MBC cell line. This cell line mimics cancer cells that overexpress MYC and Bcl2, and readily forms viable polyploid cells when cytokinesis is prevented.

### Immunofluorescent Staining:

Immunofluorescence staining was with a mouse monoclonal antibody against Histone 3 phosphorylated at Ser and has been previously described 10. Cells were cultured on coverslips in a 6-well plate, fixed with 4% paraformaldehyde and then permeabilized with 0.3% Triton X-100 in PBS. Primary antibody was detected with Texas-Red-conjugated secondary antibodies that were purchased from Jackson ImmunoResearch. After immunostaining, cells were mounted on microscope slides with 4′,6′-diamidino-2-phenylindole (DAPI)-containing Vectashield mounting solution (Vector Laboratories) for detection of fluorescence with an EVOS FL fluorescence microscope (ThermoFisher).

### Extraction, Isolation, and Purification of Compounds 1 and 2:

Samples from Corydalis longicalcarata were dried in air, chopped, and ground to fine powder in an electric grinder. Three kilograms (kg) of powder was extracted with 70% ethanol under ultrasound treatment (40 KHz) for 0.5 hours at room temperature, and then without ultrasound treatment for another 24 hours. The extraction procedure was repeated 3 times. Each time, the mixture was filtered, solvent was evaporated under vacuum using a rotary evaporator (N-1300, Tokoyo Rikakikai Co. Ltd), and the residue was dissolved in deionized water before successively partitioned against petroleum ether (PE) and ethyl acetate (EA) to obtain PE (PE, 8.5 g) and EA (EA, 9.7 g) phases, respectively.

After evaporation under vacuum, PE and EA phases were separated by column chromatograph to obtain 30 (PE1-30) and 25 (EA1-25) distinct fractions, respectively. During this process, the PE phase was loaded onto a silica gel column (100 g) and eluted manually with a step-wise gradients of petroleum ether-ethyl acetate solution (10:1, 5:1, 3:1, 2:1, 1:1, 1:2, 1:3, 1:4, 0:1, v/v) to generate the 30 fractions (PE1-30). The EA phase was subjected to low pressure preparative liquid chromatograph (SepaBean™ Machine, Santai Technologies Inc.) on a silica gel flash column (330 g). Elution was at a flow rate of 60 mL/min to produce the 25 fractions (EA1-25) with the following petroleum ether (A)-ethyl acetate (B) gradient: 0-5 min, 0→5% B; 5-20 min, 5→30% B; 20-30 min, 30→50% B; 30-60 min, 50→80% B; 60-90 min, 80→100% B.

Next, the anti-mitotic activity present in each fraction was monitored by in vitro assay. Ten fractions from the PE phase and three fractions from the EA phase were positive in these assays at a concentration of 12.5 μg/ml and these fractions were further separated using preparative silica gel TLC plates to yield 62 PE and 22 EA subfractions of further purity. TLC analyses that were carried out on preparative TLC plates (silica gel, 200 mm×200 mm×1 mm) (Yantai Xinnuo Co. Ltd) were examined with UV light at 254 and 365 nm, followed by spraying with the Dragendorff's reagent.

Eight out of the 62 PE subfractions and 3 out of the 22 EA sub-fractions tested positive for arresting cells in mitosis at a concentration of 6.25 μg/ml and fell into either of two groups based on whether their analytic HPLC spectra contain a shared peak, designated as peaks number 1 or 2 (HPLC: 1260 Infinity II LC System (Agilent); the column: Waters Xbridge C18, 4.6 mm×250 mm, 5 μm (Waters); mobile phase: A, 0.2% acetic acid-triethylamine solution (pH=5.0); B, acetonitrile. 0-5 min, 10% B; 5→10 min, 10→20% B; 10-70 min, 20→60% B; 70-75 min, 60→10% B; 75-90 min, 10% B; column temperature: 30° C.; flow rate: 1 mL/min; the injection volume: 10 μL).

Further preparative HPLC (LC-20AP, Shimadzu Corp.) for separation of these active sub-fractions was conducted on a Shimadzu Shim-pack PRC-ODS C18 column, 20 mm×250 mm, 5 μm (mobile phase: A, 0.2% acetic acid-triethylamine solution (pH=5.0); B, acetonitrile. 0-5 min, 10% B; 5-10 min, 10→20% B; 10-70 min, 20→60% B; 70-75 min, 60→10% B; 75-90 min, 10% B; column temperature: 30° C.; flow rate: 18 mL/min) and this enabled purification of compound 1 (18 mg) and compound 2 (21 mg), corresponding to the peaks 1 and 2, respectively.

### Physiochemical Properties of Compounds 1 and 2:

The structures of compounds obtained were determined by the analysis of 1H and 13C NMR spectra, ESI MS and UV absorbency spectrum. All 1H-NMR and 13C-NMR spectra were recorded in CDCl3 on a Bruker Avance III 400 MHz NMR operating at 400 MHz for 1H and 100 MHz for 13C. Chemical shifts were recorded as δ values in parts per million (ppm) and were normalized to tetramethylsilane (0.00 ppm) as an internal standard. Chemical shift multiplicities are reported as s=singlet, d=doublet, t=triplet, q=quartet and m=multiplet. Coupling constants (J) are given in Hz.

The 13C NMR spectra revealed 21 and 23 carbon atoms in compounds 1 and 2, respectively. 1H NMR spectrum of compound 2 is quite similar to that of compound 1. The specific chemical shifts in the 1H NMR at δ 1.15 (3H, s) for compound 1 and δ 1.27 (3H,s) for compound 2 imply the existence of a tertiary methyl in both compounds. Furthermore, both compounds 1 and 2 show an N-methyl singlet at δ 2.23 (3H), two doublets for two methylenedioxy groups at δ 5.97, 6.00 (4H), two aromatic singlets at δ6.65, 6.66 (2H), and a typical AB quartet at δ 6.79, 6.91 (2H, J-8.3 Hz). Compound 2 differs from compound 1 in having additional chemical shifts in 1H NMR at δ 1.86 (3H, s) and 13C NMR at δ 169.7 and 20.8, indicating the existence of an —OCOCH3 group.

LC-MS analyses were performed using the Agilent 6120 Quadrupole LC/MS instrument (Agilent Technologies). Waters Xbridge C18 columns (4.6 mm×50 mm, 5 μm) were used. Mobile phase: A, water (0.01 mol/L NH4HCO3); B, acetonitrile. Gradient: B from 5% to 100% for 1.6 min and hold 100% for 1.4 min. Column temperature: 40° C.; flow rate: 2 ml/min; the injection volume: 1 μL. NMR data together with their molecular weight of 367 and 409 revealed by MS spectrum (m/z 368.2 [M+H]+ and 410.2 [M+H]+) lead to determination of molecular formulas for compounds 1 and 2 as C21H21NO5 and C23H23NO6, respectively.

Characteristic UV absorption spectra were acquired on a Nanodrop (Thermo Fisher) and showed maximum absorption peaks at 290 nm for both compounds dissolved in methanol.

Comparison of these data with published data 4, 6 and the PubChem database led to the conclusion that compounds 1 and 2 are corynoline and acetylcorynoline, respectively. Comparative analysis of compounds 1, 2 to confirm identity was conducted against commercially available corynoline and acetylcorynoline with analytic HPLC. The purity verified by HPLC/NMR was ≥87% for the purified compounds 1 and 2 and ≥97% for commercial obtained corynoline and acetylcorynoline.

### LIST OF ABBREVIATIONS

CREST calcinosis, Raynaud's phenomenon, esophageal dysmotility, sclerodactyly, and telangiectasia.
DMSO dimethyl sulfoxide
DNA deoxyribonucleic acid
DAPI 4′,6′-diamidino-2-phenylindole
EA ethyl acetate
HPLC high performance liquid chromatography
hTERT human telomerase reverse transcriptase
IC₅₀ half maximal inhibitory concentration
MBC human medulloblastoma cells
MPLC medium pressure liquid chromatography
MTT 3-(4,5-dimethylthiazol-2-yl)-2,5-diphenyltetrazolium bromide
MYC myelocytomatosis
NMR nuclear magnetic resonance
PE petroleum ether
RPE retinal pigment epithelium
TLC thin layer chromatography
UV ultraviolet

### Example 1. Identification and Evaluation of Phytochemicals for Antimitotic Activity

A phenotypic screen of a natural products library identified an antimitotic activity in Corydalis longicalcarata rhizomes. Cellular “round-up” was used as a surrogate marker for mitotic arrest and could easily be assayed using an inverted tissue culture microscope. A crude extract library of 17,000 samples from 2,000 plant species was phenotypically screened in 96-well microplates seeded with transformed RPE-MBC cells for ability to arrest cells in mitosis. Mitotic arrest was judged by cell round-up observed under an inverted tissue culture microscope 24 hours after exposing cells to the library.

One extract, made from the rhizomes of Corydalis longicalcarata (#1779) was identified as positive, retested, and confirmed to possess multiple anti-mitotic activities in subsequent assays. These included initial provocation of the spindle checkpoint response followed by compromise, maintenance of cells in mitosis, and eventual induction of polyploidy. Additional studies were undertaken to examine the bioactive ingredient(s) from the Corydalis longicalcarata rhizome that exerts these pleiotropic effects on cell division.

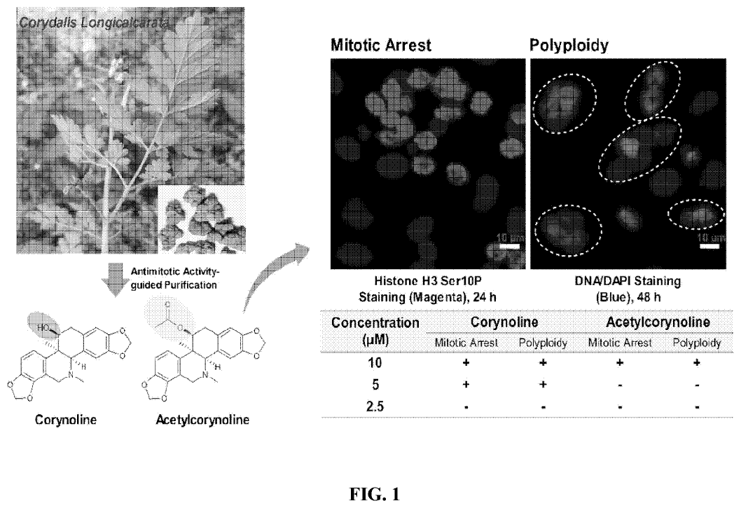

Corynoline and acetylcorynoline were purified from Corydalis longicalcarata. DAPI staining and phospho-Histone H3 Ser10 staining were conducted and examined by immunofluorescence microscopy. FIG. 1 shows that the two compounds can arrest mitosis within 24 hours of treatment and induce polyploidy at 48 hours.

### Example 2. Analysis of Compound Bioactivities of Corynoline and Acetylcorynoline

### Methods:

Cells were split, plated in appropriate culture media and allowed to adhere. After 18 hours, corynoline and acetylcorynoline were added to a cell line selected from below. At 72 h or 96 h, MTT was added and at 75 h or 99 h, the A_{570 nm} was recorded. The concentration ranged from 0.02 μM to 40 μM. Taxol was used as a positive control, which has a known IC₅₀ value of less than 0.02 μM in all tested cell lines.

The following RPE cell lines were used in these studies: RPE NEO, RPE MBH, RPE MBC, RPE MA, RPE MP, RPE MI, and RPE MYC Nick. This RPE series were derived from human retinal pigment epithelium and then stably transfected with an hTERT expression construct for immortalization. The resulting cell line was then engineered to overexpress the MYC oncogene (RPE-MYC) or a Neomycin selection marker gene (RPE-NEO). In RPE-MYC cells, a variety of other oncogenes such as Myr-AKT, BCL2, ID1 and PI3K^{E45k} were further introduced individually to generate the cell lines RPE MA, RPE MBC, RPE MI, RPE MP, respectively. As a control, H2BGFP was introduced into RPE-MYC cells to generate RPE MBH.

The following Rat1A cell lines were used in these studies: Rat1 A C, Rat1A MYC, Rat1A E2F1, and Rat1A E2F3. The cell line was then engineered as control (C), to overexpress the MYC oncogene (Rat1A-MYC), to overexpress the E2F1 oncogene (Rat1A-E2F1), or to overexpress the E2F3 oncogene (Rat1A-E2F3).

The following human cancer cell lines, representing a breadth of cancer subtypes, were used in these studies: Hela, C32, NCI-H23, HCT116 p53-/-, HCT116 wild type, MDA-MB-175IV, MDA-MB-231, MDA-MB-435, MCF-7, A431, H460, TUWI, CaoV-3, T98G, and HTB135.

### Results:

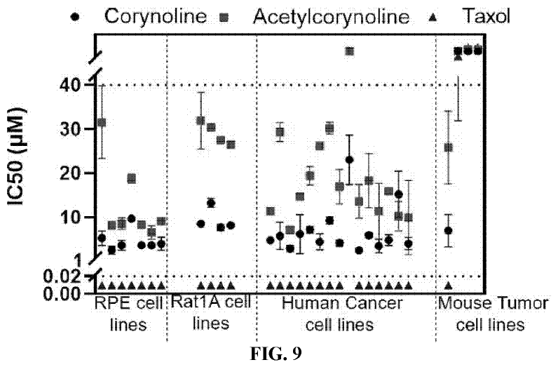

FIG. 9 shows IC₅₀ values across all of the cell lines tested. The IC₅₀ of corynoline and acetylcorynoline are almost ranged from 1˜31 μM. Taxol's IC₅₀ is a positive control, and the IC₅₀ in almost cells are <0.02 μM. The corynoline and acetylcorynoline has no significant difference on the model cell lines (oncogene-expressing RPE and Rat1A) expressed of different oncogene. The IC₅₀ of corynoline is lower than acetylcorynoline in all different cell lines, suggesting that corynoline has a better bioactivity than acetylcorynoline. The two compounds display a different pattern in one cell line. In the RPE cell line, overexpression of the Myc oncogene reduced the difference between the compounds.

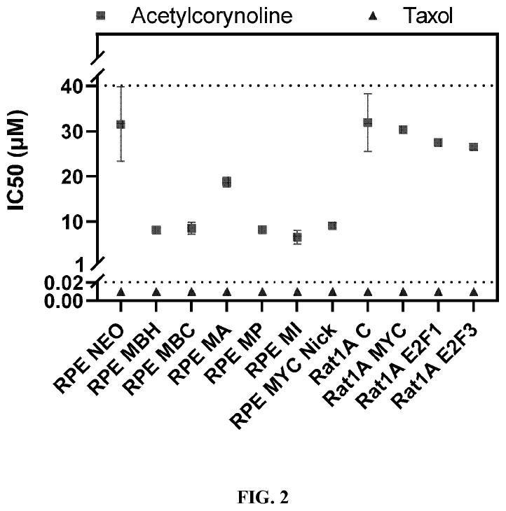

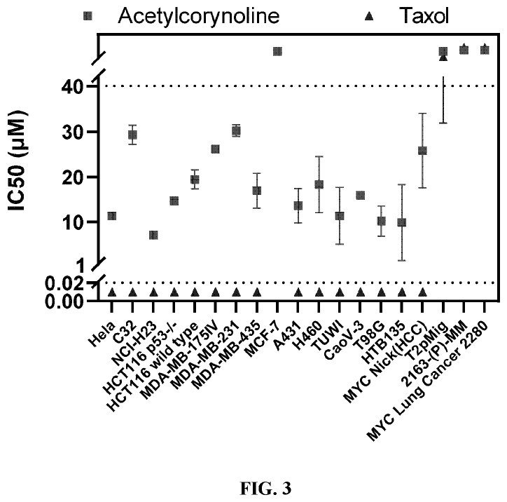

FIG. 2 shows IC₅₀ values of oncogene-expressing RPE cell lines. FIG. 3 shows IC₅₀ values of human cell lines. As shown, the majority of IC₅₀ values for acetylcorynoline fall between 1 μM to 31 μM.

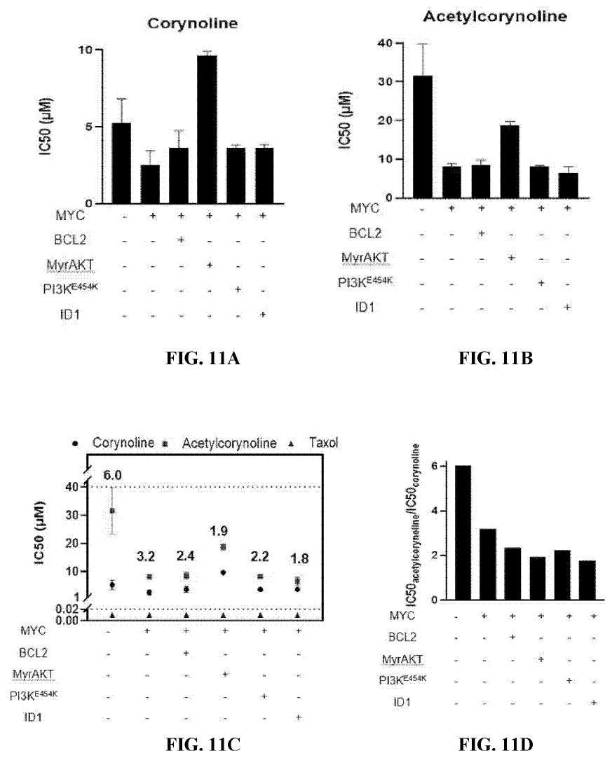

FIG. 11 shows IC₅₀ values of RPE cell lines for both corynoline and acetylcorynoline. After overexpression of different oncogenic factors, the IC₅₀ of corynoline treated cells all declined, with the exception of RPE MA. In addition, the IC₅₀ of acetylcorynoline treated cells all declined compared with the RPE NEO control, with the exception of the RPE MA cell line. These findings suggest the sensitivity of acetylcorynoline was affected by overexpression of the Myr-AKT gene.

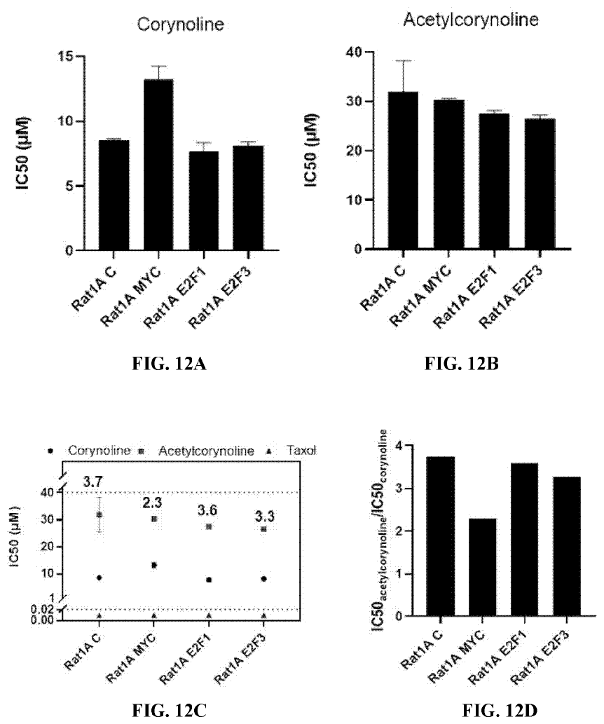

FIG. 12 shows IC₅₀ values of Rat1A cell lines. After overexpression of different oncogenes, the IC₅₀ following corynoline treatment was increased in the Rat1 A MYC cell line. The IC₅₀ of acetylcorynoline treated cells were all declined when compared to Rat1 A C, and the IC₅₀ of acetylcorynoline-corynoline data was decreased by overexpression of MYC, E2F1 or E2F3.

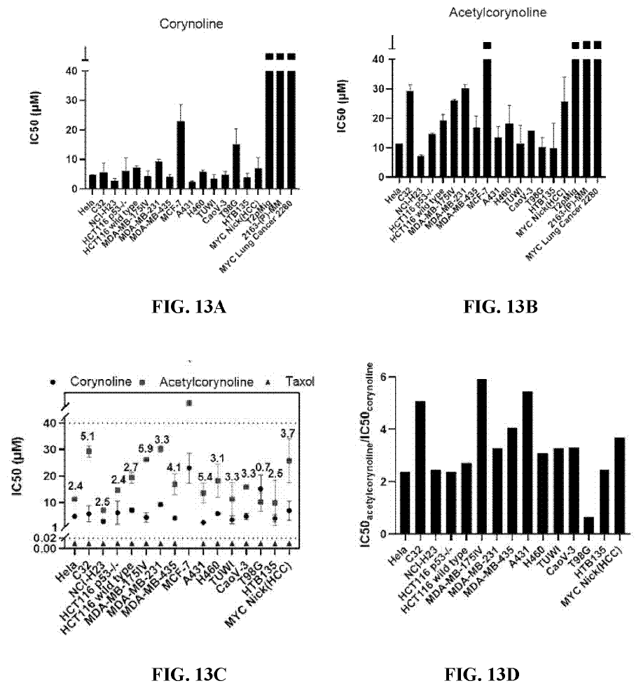

FIG. 13 shows IC₅₀ values of human cancer cell lines. With the exception of the T98G glioblastoma cell line, IC₅₀ values for acetylcorynoline were higher than those for corynoline.

### Example 3. Analysis of Spindle Pole Assembly and Early Mitosis

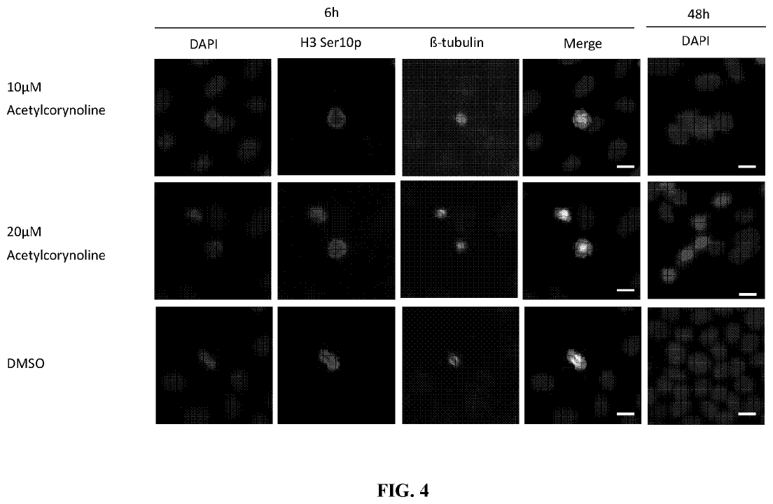

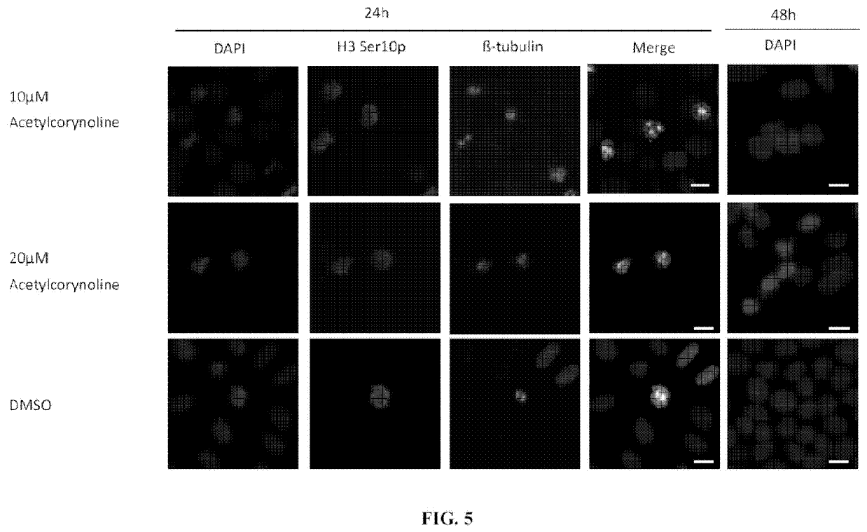

Microtubule assembly, specifically spindle pole assembly, was altered by treatment with acetylcorynoline. RPE MBC cells were treated with acetylcorynoline and Histone H3 Serine 10 Phosphorylation (Histone H3ser10p, shown in FIG. 4 and FIG. 5 in red), β-tubulin (green) and DNA (blue) was analyzed by immunofluorescence microscopy. Histone H3ser10P was used to determine the cells that were in early mitosis (e.g., prometaphase or metaphase). β-tubulin was used to examine microtubule localization and spindle assembly within the cells. FIG. 4 shows the effect of acetylcorynoline after 6 hours of treatment. Numerous mitotic cells accumulated in prometaphase with condensed DNA, as shown by the H3ser10P staining. β-tubulin staining shows a failure of bipolar spindle construction as monopolar spindles were formed. FIG. 5 shows the effect of acetylcorynoline after 24 hours of treatment. β-tubulin staining revealed that, instead of forming monopolar spindles, multipolar spindles were formed in cells that received treatment. After acetylcorynoline treatment for 48 hours, acetylcorynoline induced polyploidy, indicating that the cells eventually exited from mitosis without cytofission and consequently developed polyploidy.

### Example 4. Analysis of the Mitotic Kinetochore Using CREST Antibody

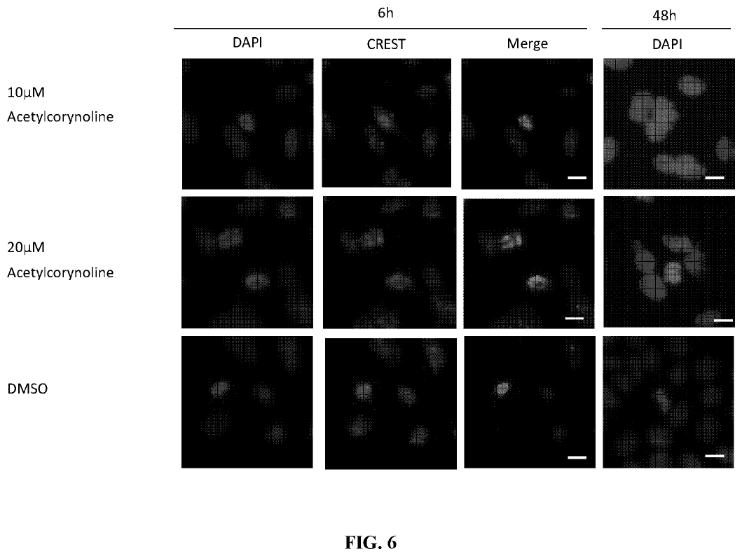

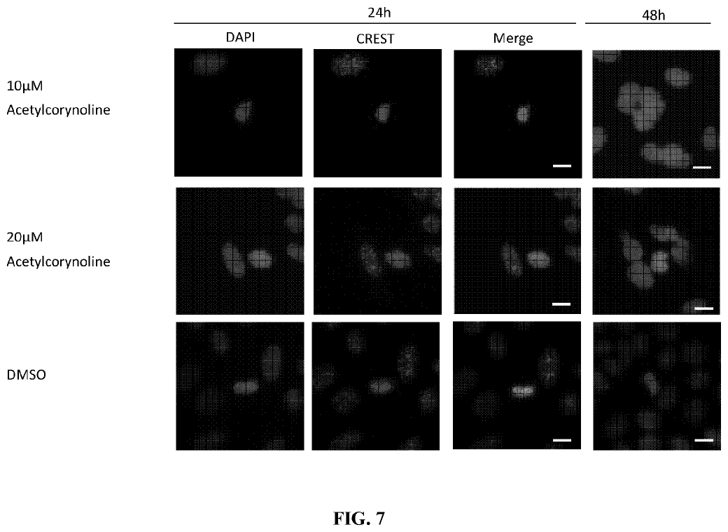

Following treatment with acetylcorynoline, RPE MBC cells were prepared for immunofluorescence analysis with CREST serum (staining shown in FIG. 6 and FIG. 7 in red). The CREST antibody specifically recognizes kinetochores in the cell, and demonstrated that kinetochore arrangement was disturbed in cells treated with acetylcorynoline. Additionally, compared to the DMSO control group at 48 hours, polyploidy was induced at both 10 μM and 20 μM acetylcorynoline, whereas cells in the control group did not have obvious cellular abnormalities. Based on these data, acetylcorynoline treatment impairs microtubule stabilization, prevents the congression of chromosomes to the cell center, and inhibits cytofission.

### Example 5. Analysis of Progression Through Mitosis by Time-Lapse Microscopy

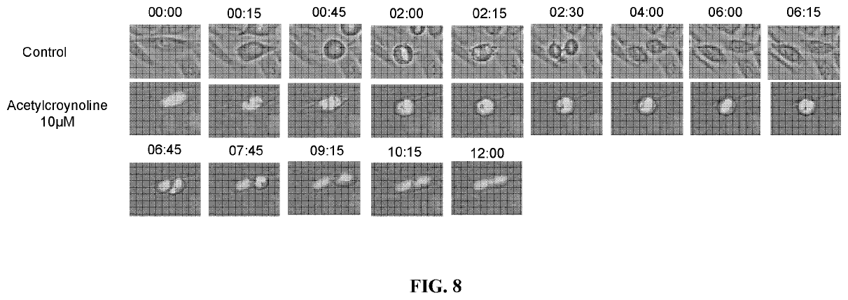

Time lapse microscopy was used to compare progression through mitosis for cells treated with acetylcorynoline and the DMSO control group, beginning at prophase. Still images are shown in FIG. 8. The time lapse experiment showed that acetylcorynoline does not affect the initiation of cytokinesis: however, acetylcorynoline prevented cytofission. Surprisingly, cells treated with acetylcorynoline spent three-times longer in early mitosis when compared to a control cell, demonstrating that acetylcorynoline stalls cells in early mitosis.

### 6. Isolation and Purification of Antimitotic Compounds From Corydalis longicalcarata Rhizomes

Isolation of Compounds From Corydalis longicalcarata Rhizomes

The success of the fractionation studies and eventual identification of bioactive components was aided by the pre-study design of a simple 96-well plate screening assay. As cells enter and become arrested in mitosis, they detach from the plate surface and appear as rounded cells with smooth surface membrane, distinct from detached cells that are undergoing programmed cell death. Simple visual screening method was used to assay thousands of extracts. It should be noted that although activity was assayed in other parts of Corydalis longicalcarata, the rhizome extract was found to contain substantial activity. This may indicate a preponderance for this portion of the plant to synthesize or store the bioactive compounds compared to other portions of the plant. To identify bioactive components that are able to interfere with mitosis, 3 kg of Corydalis longicalcarata rhizome were recollected and performed bioactivity-guided extraction, isolation, and purification. The air dried and powdered material was extracted with 70% ethanol assisted by exposure to ultrasonic waves. This liquid was then partitioned sequentially with petroleum ether (PE) and ethyl acetate (EA) to yield, in total, 55 distinct fractions by MPLC. These were again assayed for anti-mitotic activity and it was found 10 PE and 3 EA fractions with activity. Some fractions were active at lower concentrations, but not at higher. This was deemed due to Impurities that were able to alter the assay outcome and it was sought to further purify the bioactive fractions using TLC. Of the 62 PE and 22 EA TLC fractions, 11 retained mitotic activity were found in the 96-well plate assay at the lowest concentration assayed, 6.25 μg/ml. As a next step analytical HPLC was performed with the 8 positive PE sub-fractions and 3 positive EA sub-fractions. The presence of two shared peaks among the 11 sub-fractions indicated that at least two natural compounds were responsible for the antimitotic activity in the biological screening assays.

### Identification of Compounds 1 and 2 as Corynoline and Acetylcorynoline Respectively

Physiochemical analysis of compounds 1 and 2 was performed, and then asked if their chemical profiles match those of any known compounds isolated from plants of the genus Corydalis in the literature. Although compounds 1 and 2 had not matched any phytochemicals reported in Corydalis longicalcarata, they matched corynoline and acetylcorynoline, respectively, which have been identified from two other species of the genus Corydalis, Corydalis incisa 1 and Corydalis bungeana 2. Three lines of evidence supported such identification. First, as determined by the mass spectrometry, the molecular weight of compounds 1 and 2 are 367 and 409, respectively, matching those of corynoline and acetylcorynoline. Second, the two compounds are also indistinguishable from corynoline and acetylcorynoline in 1H NMR, 13C NMR and UV spectra. Third, analytic HPLC analysis demonstrated that compound 1 and the commercially obtained corynoline eluted at the same time of 27.07 min, whereas compound 2 and the commercially obtained acetylcorynoline had the exact the same elution time of 37.59 min.

### Example 8. The Pleiotropic Effects of Corynoline and Acetylcorynoline on Cell Division

Plants produce various secondary metabolites that fall into major phytochemical classes including flavonoids, tannins, terpenoids, saponins, triterpenoid saponins, alkaloids, phytosterols, carotenoids, fatty acids and essential oils 11. The main classes of metabolites that have been transformed into modern drugs include terpenes (34%), glycosides (32%), alkaloids (16%) and others (18%). Corynoline and acetylcorynoline fall into the category of benzophenanthridine alkaloids, and have been reported to have various pharmacological properties, but effects of these compounds on mitotic activity has not been reported.

The effect of the compounds on cell division was assessed using corynoline and acetylcorynoline purified either in-house or commercially. Cells treated with either of these compounds suffered similar cell-division defects. Nearly all cells entered and arrested in mitosis 24 hours after treatment (as discussed in Example 3 and shown in FIG. 5). The majority of the cells had condensed DNA without apparent alignment of chromosomes on the metaphase plate, indicating that the mitotic spindle checkpoint was activated and subsequently arrest was in prometaphase. The arrest of cells in mitosis was, however, transient. Polyploidy was observed 48 hours after the initiation of the treatment (as discussed in Example 3 and shown in FIG. 4 and FIG. 5). Thus, the mitotic spindle checkpoint was activated but failed to be maintained in the presence of corynoline or acetylcorynoline. Treated cells eventually exited from mitosis without completion of cytokinesis, leading to the formation of multinucleated cells.

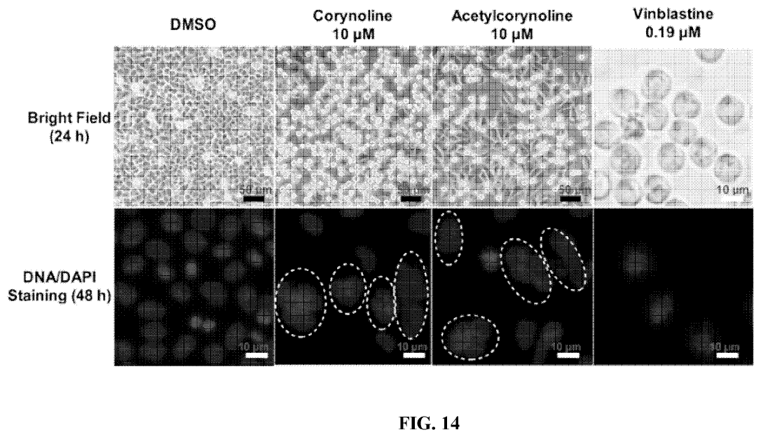

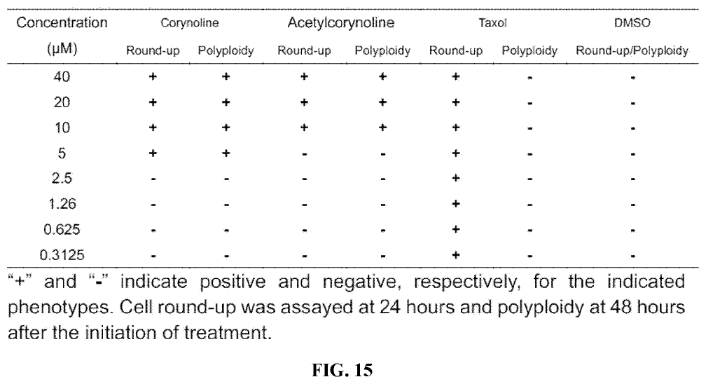

Threshold concentrations of corynoline and acetylcorynoline to elicit mitotic arrest and polyploidy were indistinguishable, indicating that disablement of the same mitotic regulator might be responsible for both effects (FIG. 15). Acetylcorynoline mimicked corynoline in eliciting both mitotic arrest and polyploidy (FIG. 14), although the minimal effective concentration of acetylcorynoline was 2-fold higher than that of corynoline (FIG. 15). Aurora-B kinase, the catalytic subunit of the chromosomal passenger protein complex, plays an essential role in coordinating chromosome segregation with cytokinesis 12. However, the effect of corynoline on cell division was not mediated by inhibition of aurora-B kinase because the phosphorylation of Histone 3 at Ser10, a surrogate of aurora-B kinase activity 13, 14 was not affected by either corynoline or acetylcorynoline (see, for example, positive staining in FIG. 4 and FIG. 5). In contrast with corynoline and acetylcorynoline, both taxol and vinblastine elicited a persistent arrest of cells in mitosis without causing accumulation of polyploid cells within 48-hour treatment (FIG. 14 and FIG. 15). Thus, corynoline and acetylcorynoline warrant further study as antimitotic therapeutics for the treatment of cancer. They possess unique anti-mitotic activities that may be advantageous to harness as a chemotherapeutic.

### Example 9. Isolation and Purification of Protopine and Chelidonine

### Protopine

Protopine was isolated from Corydalis longicalcarata by bioassay-guided purification. The procedures of purification as following: samples from dried Corydalis longicalcarata rhizomes were chopped, ground to fine powder in an electric grinder. Three kilograms (kg) of powder was extracted with 70% aqueous ethanol, and the crude extract was successively partitioned against petroleum ether (PE) and ethyl acetate (EA) to obtain PE, EA and water (WA) phases. WA phase was separated by macroporous adsorption resin column (XAD-4: AB-8, 1:1) chromatograph to obtain 10 fractions. Fr.10 (about 1 g) eluted by 100% ethanol was identified as protopine (purity, >90%).

### Chelidonine

Chelidonine was separated and enriched from Chelidonium majus L. Briefly, dried Chelidonium majus L. materials were powdered using a disintegrator. The collected powder was sifted through a 60-mesh sieve. The obtained powder (100 g) was extracted with 1000 mL of 80% aqueous ethanol under reflux for 1 h; the procedure was repeated twice. The extracted solution was filtered under vacuum; then the crude extracts were concentrated up to dryness and dissolved in deionized water to form sample solution. The sample solution was separated and enriched using D101 resin (adsorption time, 6 h) to obtain chelidonine.

The isolation and purification methods can be found, for example, in Pan et al., “Enrichment of chelidonine from Chelidonium majus L. using macroporous resin and its antifungal activity[J].” Journal of Chromatography B Analytical Technologies in the Biomedical & Life Sciences, 2017, 1070:7.

### Example 10. Analysis of Effects on Mitosis

The effect of the compounds chelidonine and protopine on cell division was assessed similarly to corynoline and acetylcorynoline above. As described in Example 6 above, as cells enter and become arrested in mitosis, they detach from the plate surface and appear as rounded cells with a smooth surface membrane. RPE-MBC cells (ectopically expressing oncogenes MYC and Bcl2) were treated with DMSO, chelidonine and protopine, and compared against controls Michael's ketone (MK) and taxol. IC50 values for each were determined and are shown in Table 1. Results were confirmed by analyzing IC50 across a broader panel of cell lines, as shown in Table 2.

TABLE 1
IC50 values for RPE-MBC or RPE-MBH cells treated with corynoline,
acetylcorynoline, chelidonine, Michael's ketone (MK) or taxol

Cell

| line | Treatment | Corynoline | Acetylcorynoline | Chelidonine | MK | Taxol | | |
|---|---|---|---|---|---|---|---|---|
| RPE- | 72 h | 8.865 | 9.940 | >40 | 0.2074 | >40 | >40 | >40 |
| MBC | | | | | | | | |
| RPE- | 72 h | 15.07 | 12.57 | 2.318 | 0.3032 | 31.38 | 22.06 | 22.69 |
| MBH | | | | | | | | |

TABLE 2
IC50 values for cell lines treated with corynoline, acetylcorynoline,
chelidonine, Michael's ketone (MK) or taxol

Cell

| line | Treatment | Corynoline | Acetylcorynoline | Chelidonine | MK | Taxol | | |
|---|---|---|---|---|---|---|---|---|
| RPE- | 72 h | 10.6 | 35.16 | 1.851 | 0.087 | 13.41 | 15.68 | 13.02 |
| A19 | | | | | | | | |
| RPE- | 72 h | 7.061 | >40 | 1.113 | 0.805 | 10.08 | 5.949 | 4.248 |
| Neo | | | | | | | | |
| IMR | 72 h | >40 | >40 | 2.433 | >40 | 6.981 | 10.84 | 5.799 |
| 90 | | | | | | | | |
| Rat1A | 72 h | 8.609 | 27.35 | 1.111 | 1.649 | 0.039 | 19.88 | 20.06 |
| 3T3 | 72 h | 12.18 | 23.02 | 1.522 | 3.48 | 0.075 | 0.062 | 0.096 |
| MEF | | | | | | | | |

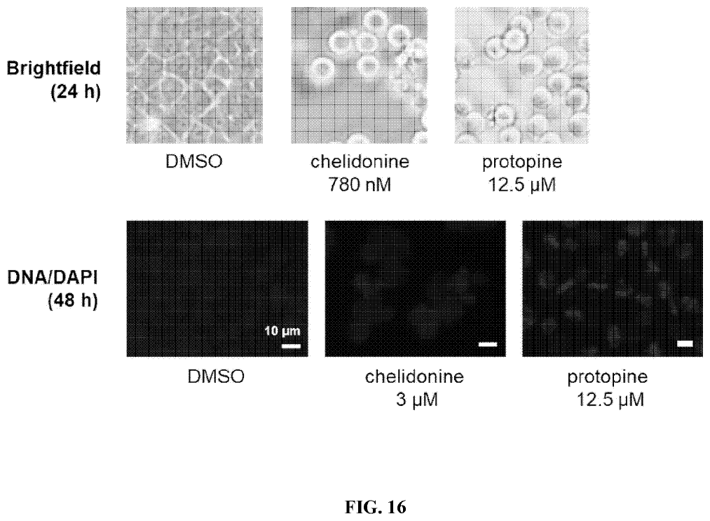

Administration of these compounds resulted in similar cell division defects. Nearly all cells entered and arrested in mitosis 24 hours after treatment (brightfield images shown in FIG. 16). The arrest of cells in mitosis was, however, transient. Polyploidy was observed 48 hours after the initiation of the treatment (immunofluorescence shown in FIG. 16). Thus, the mitotic spindle checkpoint was activated but failed to be maintained in the presence of chelidonine and protopine. Treated cells eventually exited from mitosis without completion of cytokinesis, leading to the formation of multinucleated cells.

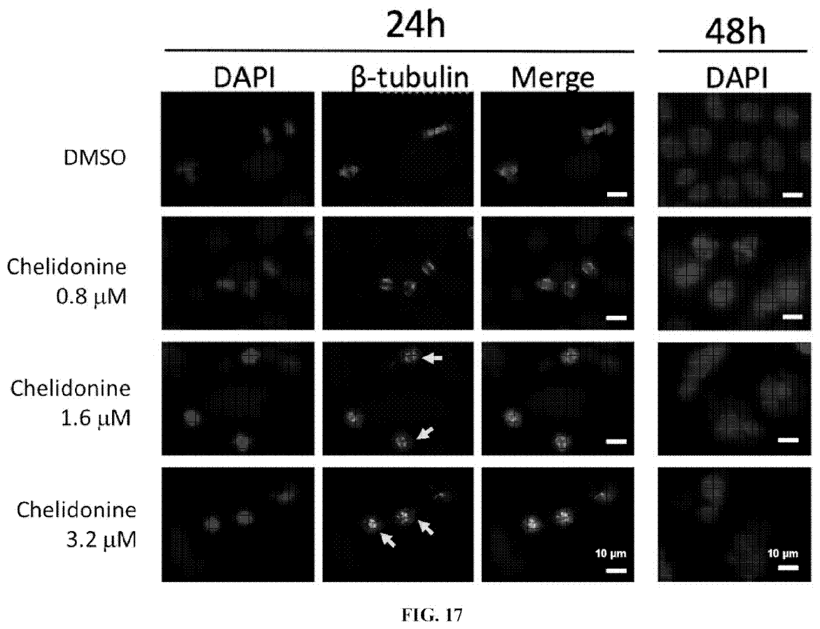

Similarly to corynoline and acetylcorynoline above, it was determined that microtubule assembly, specifically spindle pole assembly, was altered by treatment with chelidonine. MYC overexpressing RPE-MBC cells were treated with chelidonine and analyzed after 24 or 48 hours by immunofluorescence microscopy. β-tubulin was used to examine microtubule localization and spindle assembly within the cells. FIG. 17 shows the effect of acetylcorynoline after 6 hours of treatment. β-tubulin staining revealed centrosome amplification and centrosome declustering (indicated by arrows), and that, instead of forming monopolar spindles, multipolar spindles were formed in cells that received treatment. After chelidonine treatment for 48 hours, polyploidy was observed via DAPI staining, indicating that the cells eventually exited from mitosis without cytofission and consequently developed polyploidy.

These, the data demonstrate that corynoline, acetylcorynoline, chelidonine, and protopine exhibit pleiotropic effects on mitosis, through a mechanism involving dysregulation of centrosome formation, spindle assembly, and a failure of cytokinesis.

What is claimed is:

1. A method of treating cancer in a subject in need thereof, the method comprising:
administering to the subject a therapeutically effective amount of a compound of Formula I 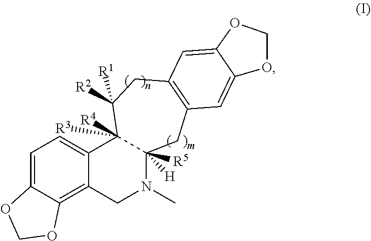
wherein:
- - - denotes no bond or a single bond;
R¹ is H;
R² is selected from —OH and —OC(O)CHCH₃;
or R¹ and R² are joined together to form, together with the carbon to which they are attached, an oxo;
R³ is selected from H and CHCH₃;
R⁴ is H or absent;
R⁵ is H or absent;
n is selected from 0 and 1;
m is selected from 0 and 1; and
provided that:
when - - - denotes no bond, both R⁴ and R¹ are H;
when - - - denotes a single bond, both R⁴ and R⁵ are absent;
when n is 0, R¹ and R² are joined together to form an oxo, R³ is H, both R⁴ and R⁵
are H, - - - is no bond, and m is 1.

2. The method of claim 1, wherein R² is —OH.

3. The method of claim 1, wherein R² is —OC(O)CH₃.

4. The method of claim 1, wherein said cancer is selected from the group consisting of ovarian cancer, lung cancer, gastric cancer, breast cancer, hepatic cancer, pancreatic cancer, skin cancer, malignant melanoma, head and neck cancer, sarcoma, bile duct cancer, cancer of the urinary bladder, kidney cancer, colon cancer, small bowel cancer, testicular embryonal 5 carcinoma, placental choriocarcinoma, cervical cancer, testicular cancer, uterine cancer, a germ cell tumor and the metastatic forms thereof.

5. The method of claim 1, wherein said cancer is selected from the group consisting of ovarian cancer, lung cancer, gastric cancer and breast cancer.

6. The method of claim 1, wherein R² is —OH and said cancer is selected from the group consisting of ovarian cancer, lung cancer, gastric cancer and breast cancer.

7. The method of claim 1, wherein R² is —OC(O)CH₃ and said cancer is selected from the group consisting of ovarian cancer, lung cancer, gastric cancer and breast cancer.

8. A method of inducing tumor cell apoptosis in a subject in need thereof, the method comprising:
administering to the subject a therapeutically effective amount of a compound of Formula I 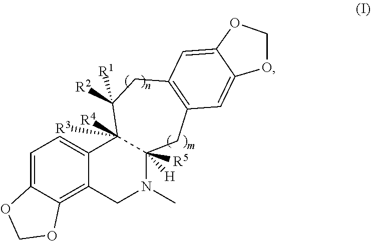
wherein:
- - - denotes no bond or a single bond;
R¹ is H;
R² is selected from —OH and —OC(O)CHCH₃;
or R¹ and R² are joined together to form, together with the carbon to which they are attached, an oxo;
R³ is selected from H and CHCH₃;
R⁴ is H or absent;
R⁵ is H or absent;
n is selected from 0 and 1;
m is selected from 0 and 1; and
provided that:
when - - - denotes no bond, both R⁴ and R⁵ are H;
when - - - denotes a single bond, both R⁴ and R⁵ are absent;
when n is 0, R¹ and R² are joined together to form an oxo, R³ is H, both R⁴ and R⁵ are H, - - - is no bond, and m is 1.

9. A method of arresting cell mitosis in a cell in a subject in need thereof, the method comprising:
administering to the subject a therapeutically effective amount of a compound of Formula I 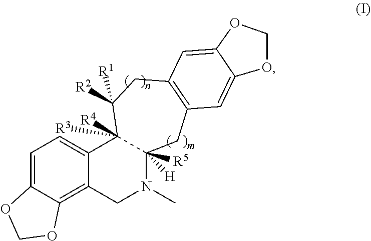
wherein:
- - - denotes no bond or a single bond;
R¹ is H;
R² is selected from —OH and —OC(O)CHCH₃;
or R¹ and R² are joined together to form, together with the carbon to which they are attached, an oxo;
R³ is selected from H and CHCH₃;
R⁴ is H or absent;
R⁵ is H or absent;
n is selected from 0 and 1;
m is selected from 0 and 1; and
provided that:
when - - - denotes no bond, both R⁴ and R⁵ are H;
when - - - denotes a single bond, both R⁴ and R⁵ are absent;
when n is 0, R¹ and R² are joined together to form an oxo, R³ is H, both R⁴ and R⁵ are H, - - - is no bond, and m is 1.

10. A method of modulating mitotic index of a cell in a subject in need thereof, the method comprising:
administering to the subject a therapeutically effective amount of a compound of Formula I 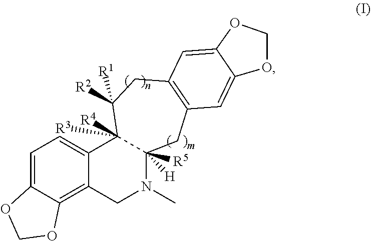
wherein:
- - - denotes no bond or a single bond;
R¹ is H;
R² is selected from —OH and —OC(O)CHCH₃;
or R¹ and R² are joined together to form, together with the carbon to which they are attached, an oxo;
R³ is selected from H and CHCH₃;
R⁴ is H or absent;
R⁵ is H or absent;
n is selected from 0 and 1;
m is selected from 0 and 1; and
provided that:
when - - - denotes no bond, both R⁴ and R¹ are H;
when - - - denotes a single bond, both R⁴ and R⁵ are absent;
when n is 0, R¹ and R² are joined together to form an oxo, R³ is H, both R⁴ and R⁵ are H, - - - is no bond, and m is 1.

11. A method of modulating a mitotic regulator, comprising administering an effective amount of a compound of Formula I 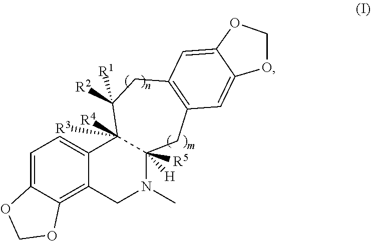
wherein:
- - - denotes no bond or a single bond;
R¹ is H;
R² is selected from —OH and —OC(O)CHCH₃;
or R¹ and R² are joined together to form, together with the carbon to which they are attached, an oxo;
R³ is selected from H and CHCH₃;
R⁴ is H or absent;
R⁵ is H or absent;
n is selected from 0 and 1;
m is selected from 0 and 1; and
provided that:
when - - - denotes no bond, both R⁴ and R⁵ are H;
when - - - denotes a single bond, both R⁴ and R⁵ are absent;
when n is 0, R¹ and R² are joined together to form an oxo, R³ is H, both R⁴ and R⁵ are H, - - - is no bond, and m is 1.

12. A method for inhibition of tumor cell growth, comprising administering an effective amount of a compound of Formula I 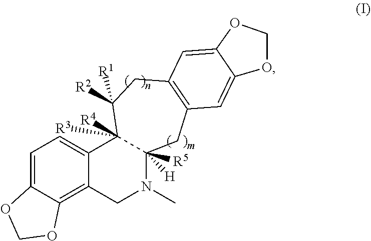
wherein:
- - - denotes no bond or a single bond;
R¹ is H;
R² is selected from —OH and —OC(O)CHCH₃;
or R¹ and R² are joined together to form, together with the carbon to which they are attached, an oxo;
R³ is selected from H and CH₃;
R⁴ is H or absent;
R⁵ is H or absent;
n is selected from 0 and 1;
m is selected from 0 and 1; and
provided that:
when - - - denotes no bond, both R⁴ and R⁵ are H;
when - - - denotes a single bond, both R⁴ and R⁵ are absent;
when n is 0, R¹ and R² are joined together to form an oxo, R³ is H, both R⁴ and R⁵ are H, - - - is no bond, and m is 1.

13. The method of any one of claims 1 to 12, wherein R² is —OH.

14. The method of any one of claims 1 to 12, wherein R² is —OC(O)CH₃.

15. The method of any one of claims 1 to 14, wherein the compound is isolated and purified from the rhizomes of Corydalis longicalcarata.

16. The method of any one of claims I to 15, wherein the compound is a mitotic inhibitor.

17. The method of any one of claims 1 to 16, wherein the compound promotes anti-mitotic activities through pleiotropic effects on cell division.

18. The method of claim 17, wherein the pleiotropic effects comprise compromise of cell division, prevention of chromosome congression, compromise of spindle checkpoint response, and blockage of cytofission.

19. The method of any one of claims 1 to 18, wherein the method is a chemotherapy.

20. The method of any one of claims 1 to 19, wherein said administering is carried out in combination with another cancer therapy.

21. A pharmaceutical composition for inhibition of tumor cell growth, comprising an effective amount of a compound of Formula I 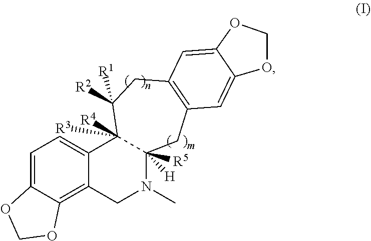
wherein:
- - - denotes no bond or a single bond;
R¹ is H;
R² is selected from —OH and —OC(O)CHCH₃;
or R¹ and R² are joined together to form, together with the carbon to which they are attached, an oxo;
R³ is selected from H and CHCH₃;
R⁴ is H or absent;
R⁵ is H or absent;
n is selected from 0 and 1;
m is selected from 0 and 1; and
provided that:
when - - - denotes no bond, both R⁴ and R¹ are H;
when - - - denotes a single bond, both R⁴ and R⁵ are absent;
when n is 0, R¹ and R² are joined together to form an oxo, R³ is H, both R⁴ and R⁵ are H, - - is no bond, and m is 1.

22. The pharmaceutical composition of claim 21, wherein R² is —OH.

23. The pharmaceutical composition of claim 21, wherein R² is —OC(O)CH₃.

24. The pharmaceutical composition of any one of claims 21 to 23, wherein the tumor cell is selected from the group consisting of hepatoma cell, an esophageal cancer cell, a cervical adenocarcinoma cell, a pancreatic cancer cell, and a leukemic cell.

25. The pharmaceutical composition of any one of claims 21 to 23 wherein the compound is isolated and purified from the rhizomes of Corydalis longicalcarata.

26. The pharmaceutical composition of any one of claims 21 to 23, wherein the compound is a mitotic inhibitor.

27. The pharmaceutical composition of any one of claims 21 to 23, wherein the compound promotes anti-mitotic activities through pleiotropic effects on cell division.

28. The pharmaceutical composition of claim 27, wherein the pleiotropic effects include compromise of cell division, prevention of chromosome congression, compromise of spindle checkpoint response, and blockage of cytofission.

29. A composition for inducing apoptosis in cancer cells, the composition comprising corynoline, acetylcorynoline, chelidonine, or protopine.

30. A method of treating cancer, the method comprising administering, to a person in need of such treatment, corynoline or acetylcorynoline in an amount sufficient to induce apoptosis and inhibit proliferation of cancer cells.
